(19) United States
(12) Patent Application Publication
(10) Pub. No.: US 2015/0238691 A1
(43) Pub. Date: Aug. 27, 2015

(54) CONTROL SYSTEMS FOR RELEASE OF MEDICATION RESPONSIVE TO JOINT ACTIVITY

(71) Applicant: Elwha LLC, Bellevue, WA (US)

(72) Inventors: Edward S. Boyden, Chestnut Hill, MA (US); Gregory J. Della Rocca, Columbia, MO (US); Philip A. Eckhoff, Kirkland, WA (US); Daniel Hawkins, Pleasanton, CA (US); Roderick A. Hyde, Redmond, WA (US); Robert Langer, Newton, MA (US); Eric C. Leuthardt, St. Louis, MO (US); Gary L. McKnight, Bothell, WA (US); Terence Myckatyn, St. Louis, MO (US); Parag Jitendra Parikh, St. Louis, MO (US); Dennis J. Rivet, Chesapeake, VA (US); Joshua S. Shimony, St. Louis, MO (US); Michael A. Smith, Phoenix, AZ (US); Clarence T. Tegreene, Mercer Island, WA (US)

(21) Appl. No.: 14/189,255

(22) Filed: Feb. 25, 2014

(51) Int. Cl. A61M 5/172; A61F 2/30; A61M 31/00

(52) U.S. Cl. CPC A61M 5/1723; A61M 31/002; A61F 2/30; A61M 2005/1726; A61M 2210/02; A61M 2205/3306; A61M 2205/3317; A61M 2205/3592; A61M 2205/502; A61F 2250/0068; A61F 2250/0096

(57) ABSTRACT

In some embodiments, a system for responsive release of a medicament in an artificial joint region includes: an implantable sensor unit including at least one sensor, the implantable sensor unit configured to be implanted in an artificial joint region; a responsive release control unit including an electronic controller and memory, the responsive release control unit configured to receive signals from the implantable sensor unit and to send signals to an implantable medicament release unit; and the implantable medicament release unit, including a reservoir and a controllable release unit attached to the reservoir, the controllable release unit configured to provide access to the reservoir in response to signals from the responsive release control unit, the implantable medicament release unit configured to be implanted in the artificial joint region.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In one aspect, a system includes but is not limited to a system for responsive release of a medicament in an artificial joint region, including: an implantable sensor unit including at least one sensor, the implantable sensor unit configured to be implanted in an artificial joint region; a responsive release control unit including an electronic controller and memory, the control unit configured to receive signals from the implantable sensor unit and to send signals to an implantable medicament release unit; and the implantable medicament release unit, including a reservoir and a controllable release unit attached to the reservoir, the controllable release unit configured to provide access to the reservoir in response to signals from the control unit, the implantable medicament release unit configured to be implanted in the artificial joint region. In addition to the foregoing, other system aspects are described in the claims, drawings, and text forming a part of the disclosure set forth herein.

In one aspect, a system includes but is not limited to a system for responsive release of a medicament in an artificial joint, including: at least one fiducial unit configured to attach to a first region of a first component of an artificial joint; an implantable sensor unit configured to attach to a second region of a second component of the artificial joint, the implantable sensor unit including at least one sensor configured to sense a position relative to the at least one fiducial unit; a responsive release control unit including an electronic controller and memory, the responsive release control unit configured to receive signals from the implantable sensor unit and to send signals to an implantable medicament release unit; the implantable medicament release unit, including at least one reservoir, and at least one controllable release unit configured to respond to signals from the control unit.

In one aspect, a system includes but is not limited to a responsive release control unit for a medicament in an artificial joint, including: circuitry configured to accept information relating to motion of an artificial joint in an individual; circuitry configured to save the accepted information in memory within an artificial joint motion history; circuitry configured to form a comparison of the artificial joint motion history with preset parameters of the motion of the artificial joint for the individual; circuitry configured to determine if the comparison exceeds a preset limit for the individual; and circuitry configured to send a signal to an implanted medicament release unit in response to the determination.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is an illustration of an artificial hip joint.

FIG. 2 is an illustration of an artificial hip joint.

FIG. 3 is an illustration of an artificial knee joint.

FIG. 4 is an illustration of an artificial shoulder joint.

FIG. 5 is an illustration of an artificial hip joint system.

FIG. 6 is an illustration of an artificial knee joint system.

FIG. 7 is an illustration of an artificial hip joint.

FIG. 8 is an illustration of an artificial hip joint.

FIG. 9 is an illustration of an artificial knee joint.

FIG. 10 is an illustration of an artificial shoulder joint.

FIG. 11 depicts aspects of a responsive release control unit for a medicament in an artificial joint.

FIG. 12 is an illustration of an artificial hip joint.

FIG. 13 is an illustration of an artificial knee joint.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The use of the same symbols in different drawings typically indicates similar or identical items unless context dictates otherwise.

Over time and use, artificial joints are subject to osteolysis and bone resorption which may cause loosening of the implanted joint and require additional surgery to reset the prosthesis. In some situations, wear debris from the artificial joint itself can lead to inflammatory response in the patient and related problems with the artificial joint. See e.g., Agarwal, “Osteolysis—Basic Science, Incidence and Diagnosis,” Current Orthopaedics 18:220-231 (2004) and Collier et al., “Osteolysis After Total Knee Arthroplasty: Influence of Tibial Baseplate Surface Finish and Sterilization of Polyethylene Insert” J. Bone Joint Surg. 87-A: 2702-2708, (2005), which are each incorporated herein by reference. Greater load and motion of an artificial joint over time increases the possibility of wear, osteolysis, and inflammation, with the eventual possibility of further surgical intervention being required.

Artificial joint systems described herein include sensors to monitor load and/or motion of the joint over time, and controllers with the capacity to receive information from the sensors and compare it to stored data and standards. Artificial joint systems also include at least one implantable medicament release unit, including a reservoir and a controllable release unit attached to the reservoir, the controllable release unit configured to release medicament to the joint region in response to signals from the controller. Artificial joint systems include controlled release of medicament(s) into the artificial joint region from implanted reservoirs in response to data from associated load and/or motion sensors. For example, in some embodiments a controller can be configured to receive information from a load sensor and a motion sensor affixed to an artificial joint, and to compare the received information to historical data as well as to standards stored in memory in the controller. The controller can also send signals to a controllable release unit attached to a medicament reservoir. For example, if data from one or more sensors is consistent with cumulative or total load or motion above a preset threshold, a controller can send a signal to a controllable release unit that initiates the controllable release unit to release osteolysis-inhibiting medicament from the reservoir. For example, if data from one or more sensors is consistent with cumulative load or motion above a preset threshold during a predetermined period of time, a controller can send a signal to a controllable release unit that initiates the controllable release unit to release inflammation-inhibiting medicament from the reservoir.

In some embodiments, a controller can be configured to accept data regarding usage of an artificial joint, including motion (e.g. motion above a minimal threshold, and/or total range of motion) and/or load (e.g. total mass, and/or load at a particular angle) and to calculate one or more usage values based on the accepted data. For example, in some embodiments a controller can be configured to calculate one or more total load values on a joint based on accepted data, such as cumulative load, load during a predetermined time period, or maximum load reached. For example, in some embodiments a controller can be configured to calculate motion values in a joint based on accepted data, such as the number of motion cycles during an interval of time, the maximum range of motion of the joint, the minimum range of motion of the joint, or the average range of motion of the joint. In some embodiments, a controller can be configured to calculate one or more values based on accepted data relating to both load and motion, for example maximum load during maximum range of motion of the joint, minimum load during maximum range of motion of the joint, average load during motion of the joint, average load at various motion positions of the joint, etc. In some embodiments, a controller is attached to a clock unit, and is configured to calculate values like those stated above during a period of time and/or time interval. In some embodiments, a controller is configured to compare calculated values for different time periods (e.g. by day, week, or month). In some embodiments, a controller includes memory and is configured to store calculated values such as those described above and to compare newly calculated values to historical or saved values. For example, a controller can be configured to calculate a weekly average load for a joint, and to compare that calculated value to a historical average load for that joint each week. A controller can initiate release of one or more medicaments in relation to one or more calculated values.

A “medicament,” as used herein, includes medications, therapeutics or treatments targeted to reduce the physiological reaction to artificial joint use, including load and motion, and to prolong the use of the joint. In some embodiments, a medicament includes anti-inflammatory agents, such as small-molecule drugs targeting aspects of an inflammation cascade in the joint, for example cytokines, and cytokine-derived molecules. In some embodiments, a medicament includes molecules known to bind with reactive oxygen species that can be created by excess wear in an artificial joint. In some embodiments, a medicament includes analgesics directed to minimize pain associated with joint wear. For example, depending on the embodiment, medicament stored for release by a reservoir can include: antibiotics, analgesics, or medicaments selected to improve the stability of the artificial joint. For example, medicaments selected to improve the stability of the artificial joint can include one or more of: antibody treatments, cytokine treatments, small molecule drugs, or proteins selected to mitigate the process of osteolysis in the artificial joint over time and use.

A “reservoir,” as used herein, refers to a device configured to store one or more medicaments without release over a period of time, and then release the one or more medicaments in response to a signal from a controllable release unit. In some embodiments, a reservoir includes a medicament reservoir. In some embodiments, a reservoir includes a reservoir configured to store a single medicament. In some embodiments, a reservoir includes a reservoir configured to store multiple medicaments for release from the same signal from a controllable release unit. In some embodiments, a reservoir includes a reservoir configured to store multiple medicaments, and is configured to release a first medicament at a first time and at least one subsequent medicament at a subsequent time. In some embodiments, a reservoir stores one or more medicaments within one or more chambers internal to the reservoir. For example, a reservoir can include microreservoirs with electronic control of drug release. See e.g., U.S. Patent Application No. 2007/0016163 “Medical and Dental Implant Devices for Controlled Drug Delivery,” by Santini, Jr. et al. published on Jan. 18, 2007 which is incorporated herein by reference. For example, a reservoir can include a reservoir configured for use with hollow microneedle arrays during release of the medicament. See, e.g., McAllister et al., “Microfabricated Needles for Transdermal Delivery of Macromolecules and Nanoparticles: Fabrication Methods and Transport Studies,” Proc. Natl. Acad. Sci. USA, 100: 13757-13760, (2003) which is incorporated herein by reference.

Artificial joints, as used herein, include but are not limited to: artificial hip joints, artificial knee joints, artificial shoulder joints, and artificial ankle joints. The artificial joints can include total or partial joint replacements, depending on the embodiment. In some embodiments, the artificial joint systems are components that attach to regions of a joint that are otherwise left intact. For example, some embodiments include components that attach to the internal surfaces of a joint in a manner that does not impede movement but does provide sensor data (e.g. load and/or motion data) to inform controlled release of medicament in another region of the joint. For example, a sensor unit can be affixed to a cartilage or bone face of a joint while an implantable medicament release unit of the same system is embedded within an artificial portion of the joint.

In some embodiments, a system for responsive release of a medicament in an artificial joint region includes: an implantable sensor unit including at least one sensor, the implantable sensor unit configured to be implanted in an artificial joint region; a responsive release control unit including an electronic controller and memory, the control unit configured to receive signals from the implantable sensor unit and to send signals to an implantable medicament release unit; and the implantable medicament release unit, including a reservoir and a controllable release unit attached to the reservoir, the controllable release unit configured to provide access to the reservoir in response to signals from the control unit, the implantable medicament release unit configured to be implanted in the artificial joint region.

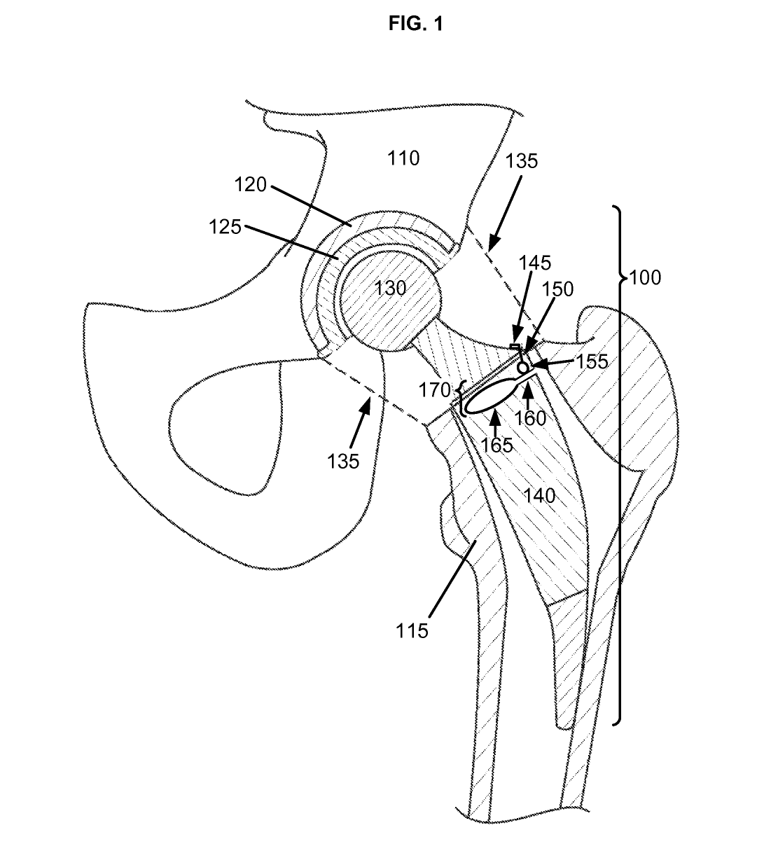

For example, FIG. 1 illustrates an artificial joint 100 prosthesis in situ within an individual patient. The artificial joint 100 is a hip joint prosthesis depicted in partial cross-section in order to illustrate interior aspects of the joint and joint prosthesis not visible from an external view. The artificial joint 100 includes an outer acetabular cup 120 surrounding an acetabular liner 125. The acetabular cup 120 is affixed to the pelvis 110 of a patient. The artificial hip joint 100 also includes a set of femoral components, including a femoral shaft 140 attached to a femoral ball 130. The femoral shaft 140 is partially positioned within the interior of a femur 115 of the patient. It is expected that a femoral shaft 140 of an artificial hip joint will be affixed to the femur 115 using standard techniques. The femoral ball 130 is attached to the femoral shaft 140 and positioned within the acetabular liner 125. A membrane 135 surrounds and encapsulates the joint, including joint fluid within the joint region. The membrane 135 is attached to both the pelvis 110 and the femur 115 of the patient, substantially surrounding the artificial joint. The membrane 135 substantially defines the boundary of the artificial joint region. In some embodiments, systems are positioned and configured to responsively release medicament(s) into an artificial joint region by release into the joint fluid surrounded by the membrane 135 around the artificial joint 100.

As shown in FIG. 1, the artificial joint 100 includes an implantable sensor unit 145 attached to the femoral shaft 140. The sensor unit is configured and positioned to sense physical changes arising from use of the artificial joint, such as load during use and motion, or range of motion, during each use. Some embodiments include an implantable sensor unit that is configured to be affixed to the artificial joint region. For example, in some embodiments an implantable sensor unit is directly affixed to a component of an artificial joint, for example attached with a fastener or an adhesive to a surface of the artificial joint. For example, in some embodiments an implantable sensor unit is included on a rim, liner, cover or appendage to the joint. For example a sensor unit can be affixed to a thin plate inserted within the wear surfaces of a joint and configured to detect load and/or motion at the wear surfaces. For example, in some embodiments an implantable sensor unit is included on a disk configured to surround one or more components of an artificial joint, such as the stem of a femoral component of an artificial hip joint. Some embodiments include an implantable sensor unit that is integral to an artificial joint component. For example, one or more components of a motion sensor and/or a load sensor can be integrated into the structure of an artificial joint, such as during fabrication of the artificial joint component. A component of an artificial joint configured for use with a system for responsive release of a medicament in an artificial joint region can include one or more surfaces and/or interior regions configured for addition of a sensor unit, for example including one or more hollows or indentations including a surface configured to reversibly mate with a surface of a sensor unit to form a combined unit with a size and shape suitable for use as an artificial joint within a patient.

A sensor unit 145 such as illustrated in FIG. 1 can include, for example, a load sensor positioned and configured to respond to the load carried by the artificial joint during use by the patient. For example, an implantable sensor unit attached to the edge of the stem of a femoral component, or the ball of a femoral component, of an artificial hip joint can include a load sensor that includes a microcantilever configured for detecting loads applied to the joint. See e.g., U.S. Pat. No. 7,097,662 “In-Vivo Orthopedic Implant Diagnostic Device for Sensing Load, Wear and Infection” issued to Evans, III et al. on Aug. 29, 2006 which is incorporated herein by reference. In some embodiments, a sensor unit includes a motion sensor positioned and configured to respond to the motion, and/or the range of motion, of the artificial joint during use by a patient. For example, a sensor unit attached to the edge of the stem of a femoral component, or the ball of a femoral component, of an artificial hip joint can include a motion sensor including at least one ultrasound sensor and at least one accelerometer. The motion sensor can be configured to localize and track the motion of bone joints and surrounding ligaments and soft tissue. See e.g.: U.S. Patent Application No. 2010/0198067 “Noninvasive Diagnostic System” by Mahfouz et al. published Aug. 5, 2010; U.S. Pat. No. 7,918,887 “Body Parameter Detecting Sensor and Method for Detecting Body Parameters” to Roche; and U.S. Pat. No. 5,533,519 “Method and Apparatus for Diagnosing Joints” to Radke et al., which are each incorporated herein by reference. In some embodiments, a sensor unit includes both a motion sensor and a load sensor. In some embodiments, a sensor unit includes a position sensor, configured to detect a position of the artificial joint. For example, a sensor unit can include at least one position sensor oriented and configured to detect the relative position of components of an artificial joint during use of the joint by a patient. For example, a sensor unit can include at least one position sensor configured to detect the relative position of a fiducial unit affixed to a component of the artificial joint. In some embodiments, a sensor unit includes a distance sensor, positioned and configured to detect the distance between components of an artificial joint. For example, a sensor unit can include at least one distance sensor configured to detect the distance to a fiducial unit affixed to a component of the artificial joint. The sensor unit is configured to send data to a responsive release control unit within the system. In some embodiments, a sensor unit includes a transmitter. For example, a sensor unit can include an ultrasound transmitter. For example, a sensor unit can include a transmission unit that is configured to send electrical signals encoding data along a wire.

A system for responsive release of a medicament in an artificial joint region also includes a responsive release control unit. For example, FIG. 1 illustrates a responsive release control unit 155 positioned within the femoral shaft 140 of the artificial joint 100. In some embodiments, a responsive release control unit includes a controller and memory, which in some embodiments is an electronic controller and electronic memory. A responsive release control unit is configured to receive signals from the implantable sensor unit and to send signals to an implantable medicament release unit within the system. For example, the responsive release control unit 155 shown in FIG. 1 is connected to the sensor unit 145 with a wire connection 150. In some embodiments, a responsive release control unit is connected to a sensor unit with a wireless connection. For example, in some embodiments the responsive release control unit is connected to a sensor unit via ultrasound transmissions. A responsive release control unit is configured to receive signals transmitted from the sensor unit. For example, a responsive release control unit can be connected to a sensor unit with a wire connection, the responsive release control unit including circuitry configured to receive signals including data from an attached sensor unit and to process the received data. In some embodiments, a responsive release control unit includes a receiver and a signal processor. In some embodiments, a responsive release control unit includes an antenna.

In some embodiments, the responsive release control unit of a system for responsive release of a medicament in an artificial joint region includes memory, such as electronic memory. In some embodiments, the responsive release control unit includes circuitry, for example, one or more processors. In some embodiments, the responsive release control unit includes a clock function, or circuitry configured to record elapsed time. In some embodiments, the memory includes information relevant to the release of medicament in response to use of the artificial joint over time. For example, a responsive release control unit can include one or more look-up tables in memory, the look-up tables including predetermined parameters of joint use over time that are associated with osteolysis which may be treated with one or more medicaments. In some embodiments, a responsive release control unit includes look-up tables of load and/or motion over time, the look-up tables also including the threshold values of both load and motion combined that are predetermined to be suitable for medicament intervention. For example, an embodiment of an artificial joint can be estimated in advance of use in a patient to begin to show signs of osteolysis, on average, after 20 years assuming use in a routine manner. However, an individual patient who has a lifestyle profile that suggests greater wear on the joint than usual will occur (e.g. a patient with long-term athletic goals, or a patient of above-average mass) can utilize a version of the artificial joint that includes a system for responsive release of a medicament in an artificial joint region. For example, an artificial joint configured for use with an athletic patient can include at least one motion sensor, and a responsive release control unit included with the system can include look-up tables with one or more threshold values of motion within predetermined time ranges wherein release of a medicament is considered appropriate by a physician to minimize the potential for osteolysis. For example, an artificial joint configured for use with a patient of above-average mass can include at least one load sensor, and a responsive release control unit included with the system can include look-up tables with one or more threshold values of load within predetermined time ranges wherein release of a medicament is considered appropriate by a physician to minimize the potential for osteolysis.

A responsive release control unit of a system for responsive release of a medicament in an artificial joint region is configured to send signals to an implantable medicament release unit. For example, in some embodiments a responsive release control unit includes a transmitter. For example, in some embodiments a responsive release control unit includes a transmitter and a signal processor. For example, in some embodiments a responsive release control unit is attached to an implantable medicament release unit with a wire connection, the responsive release control unit including circuitry configured to send signals to the implantable medicament release unit in response to data accepted from one or more sensors within the artificial joint. In some embodiments, a responsive release control unit is configured to send wireless signals to an implantable medicament release unit in the artificial joint, and the implantable medicament release unit is configured to receive those signals.

A system for responsive release of a medicament in an artificial joint region includes an implantable medicament release unit. An implantable medicament release unit is configured to be implanted in the artificial joint region. In some embodiments, an implantable medicament release unit is configured to be positioned so as to release at least one medicament at a location adjacent to a bone/prosthesis interface. In some embodiments, an implantable medicament release unit is configured to be positioned so as to release at least one medicament into the joint fluid of an artificial joint. In some embodiments, a system for responsive release of a medicament in an artificial joint region includes an implantable sensor unit configured to attach to a first location on a first component of the artificial joint, and an implantable medicament release unit that is configured to attach to a second location on a second component of the artificial joint. For example, a system for responsive release of a medicament in an artificial joint region can include an implantable sensor unit configured to attach at a position adjacent to a load-bearing surface of the artificial joint, and an implantable medicament release unit positioned to release one or more medicaments at a position adjacent to the artificial joint/bone interface. See, e.g., FIG. 1. Some embodiments include a fiducial marker configured to be attached to the joint at a first location on a first component of the artificial joint, and an implantable sensor unit configured to be attached to the artificial joint at a second location on a second component, wherein the implantable sensor unit includes a sensor configured to detect a relative position of the fiducial marker.

An implantable medicament release unit includes a reservoir and a controllable release unit attached to the reservoir. The controllable release unit is configured to provide access to the reservoir in response to signals from the control unit. In some embodiments, an implantable medicament release unit is modular, for example fabricated to allow a reservoir to be selectively attached to different types of controllable release units. In some embodiments, an implantable medicament release unit is modular, for example fabricated to allow a controllable release unit to be selectively attached to different types of reservoirs.

For example, as illustrated in FIG. 1, in some embodiments a responsive release control unit 155 is directly attached to an implantable medicament release unit 170. The implantable medicament release unit 170 includes a reservoir 165 and a controllable release unit 160 attached to the reservoir 165. The controllable release unit 160 illustrated in FIG. 1 is positioned to selectively open a conduit between the interior of the attached reservoir 165 and a location adjacent to an interface between the femur bone 115 and the femoral shaft 140 of the artificial joint 100. In the embodiment illustrated, the controllable release unit 160 is configured to selectively open the conduit in response to signals received from the responsive release control unit 155. Once the conduit is opened, one or more medicaments present in the interior of the reservoir move towards the bone/prosthesis interface through diffusion.

An implantable medicament release unit includes a controllable release unit, configured to provide access to the reservoir in response to signals from the control unit. In some embodiments the controllable release unit sends one or more signals to a reservoir, the signals configured to cause the release of one or more medicaments by the reservoir. For example, in some embodiments a controllable release unit includes one or more electrical pulse generators. For example, in some embodiments a controllable release unit includes one or more optical pulse generators. For example, in some embodiments a controllable release unit includes one or more devices positioned to reversibly produce physical pressure on the reservoir.

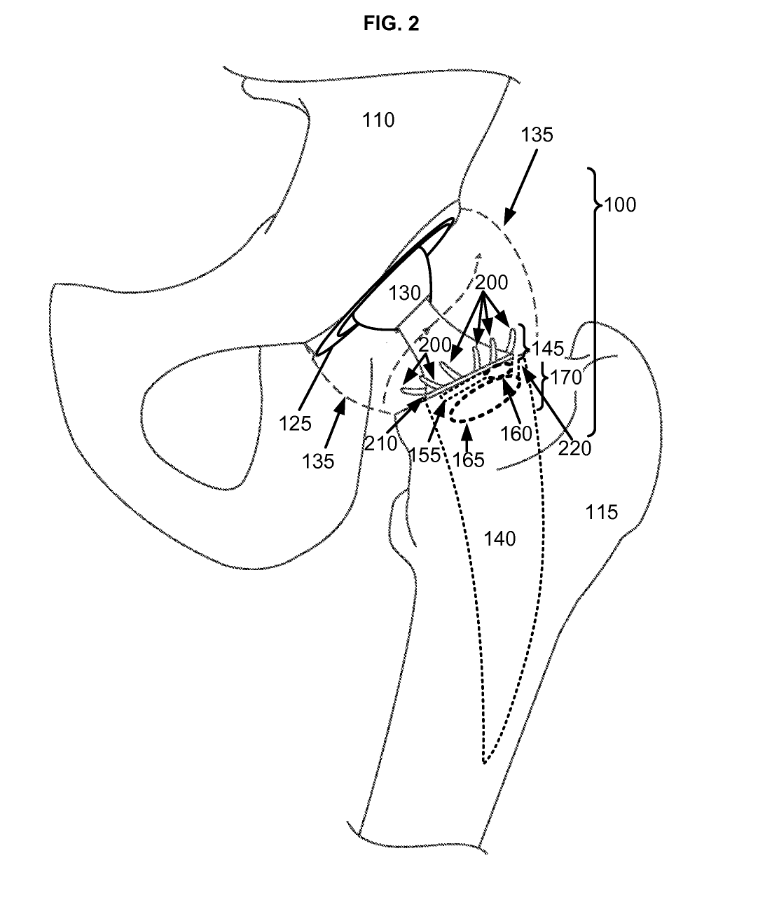

FIG. 2 illustrates an embodiment of a system for responsive release of a medicament in an artificial joint region. The artificial joint 100 shown in FIG. 2 is a prosthetic hip joint. The artificial joint 100 includes an acetabular liner 125 affixed to a pelvis 110 of a patient. The acetabular liner 125 is attached to an acetabular cup (not shown in the view of FIG. 2). The artificial joint 100 includes a femoral component 140 positioned partially within the femur 115 of the patient, and attached to the femur with usual means. A femoral ball 130 is affixed to the stem of the femoral component 140. The femoral ball 130 includes an outer surface of a size and shape to reversibly mate with the interior surface of the acetabular liner 125. A membrane 135 is attached to both the pelvis 110 and the femur 115, the membrane 135 substantially enclosing the artificial joint 100. The membrane 135 substantially delineates the boundary of the artificial joint region. The membrane 135 substantially confines joint fluid within the artificial joint, so that the joint fluid moves within the confines of the membrane 135 boundary during use of the joint (as illustrated by the dotted lines across the center of the artificial joint region in FIG. 2).

The embodiment shown in FIG. 2 includes an implantable sensor unit 145 encircling the femoral component 140 at a position adjacent to the interface between the femoral component 140 and the femur 115. The implantable sensor unit 145 illustrated includes a base 210 attached to a plurality of protrusions 200. In the embodiment illustrated, the implantable sensor unit 145 includes a plurality of protrusions 200 which extend into the artificial joint region at positions around the implantable sensor unit 145. The plurality of protrusions 200 are configured as substantially linear structures, with a first end projecting into the joint fluid within the artificial joint region and a second end affixed to a base 210 of the implantable sensor unit 145 surrounding the femoral component 140. Depending on the embodiment, the protrusions can be fabricated from an implant-compatible plastic, and attached to a base fabricated from similar material. Depending on the embodiment, the protrusions can be fabricated from an implant-compatible metal, and attached to a base fabricated from similar material. In some embodiments, the protrusions are attached to the base with mechanical fasteners, such as hinges or similar structures. In some embodiments, the protrusions are fabricated to be integral to the base, such as fabricated from a single piece of implant-compatible plastic. In the embodiment illustrated, each of the protrusions 200 of the implantable sensor unit 145 include a cantilever sensor which is connected to the base 210 of the implantable sensor unit 145. The base 210 of the implantable sensor unit 145 is connected to a responsive release control unit 155. In the embodiment shown, information from the cantilever sensors embedded in each of the protrusions 200 is transmitted through the base 210 to the responsive release control unit 155 through internal wires (not visible in the view of FIG. 2). The view of FIG. 2 includes, for purposes of illustration, protrusions that will not be to scale in all embodiments. Some embodiments include protrusions, for example, of between approximately 2-7 mm in length and approximately 1-2 mm in width. Some embodiments include protrusions, for example, of between approximately 5-10 mm in length and approximately 2-4 mm in width.

The protrusions 200 of the implantable sensor unit 145 can generate data regarding the amount of motion of the joint, which can be integrated with historical data within the responsive release control unit 155. For example, the number of times that the joint moves in a period of time, such as 24 hours, 48 hours, 72 hours, etc. can be calculated and recorded as either data from a particular time period or utilized as the basis for calculating further values, such as time-based averages or cumulative motion for the joint. In some embodiments, the amount of motion of the joint can be recorded and calculated, such as the number of protrusions utilized in every motion. For example, at some times all of the protrusions may be bent by the joint motion, as transmitted through the joint fluid, and at other times only a portion or percentage of the protrusions may be bent. This percentage involvement of the protrusions can be utilized by the system to provide weighted information about the amount of motion made by the artificial joint during movement by the patient. In some embodiments, the amount of motion of the joint can be recorded and calculated, such as the degree of motion for each of the protrusions utilized in every motion. For example, the degree of flex or bending of each protrusion can be recorded during motion by the patient, and used as a basis for calculations regarding the amount of motion of the joint to form weighted information about the amount of motion made by the artificial joint during movement by the patient. These values can serve as a basis for calculating cumulative motion of the joint by the processor within the responsive release control unit.

In the embodiment shown in FIG. 2, the cantilever sensors embedded in each of the protrusions 200 are connected to the base 210. Data from the sensors is transmitted from the base 210 to the attached responsive release control unit 155. The embodiment illustrated in FIG. 2 also includes an implantable medicament release unit 170. The implantable medicament release unit 170 includes a controllable release unit 160, the controllable release unit positioned and configured to receive transmissions from the responsive release control unit 155. In the embodiment shown in FIG. 2, the responsive release control unit 155 is physically attached to the controllable release unit 160. Internal wires carry transmission signals from the responsive release control unit 155 to the controllable release unit 160. The controllable release unit 160 is attached to a reservoir 165. The reservoir 165 contains and holds one or more medicaments within its interior region until a release signal is sent from the controllable release unit 160. In the embodiment shown in FIG. 2, a conduit 220 is positioned between the reservoir 165 and a location adjacent to the interface between the bone and the femoral component 140 of the artificial joint 100. In response to a signal from the controllable release unit 160, medicament is released from the reservoir 165 to diffuse through the conduit 220. For example, in some embodiments the conduit 220 includes a plug configured to disperse in response to an electric potential across the conduit 220 generated by the controllable release unit 160.

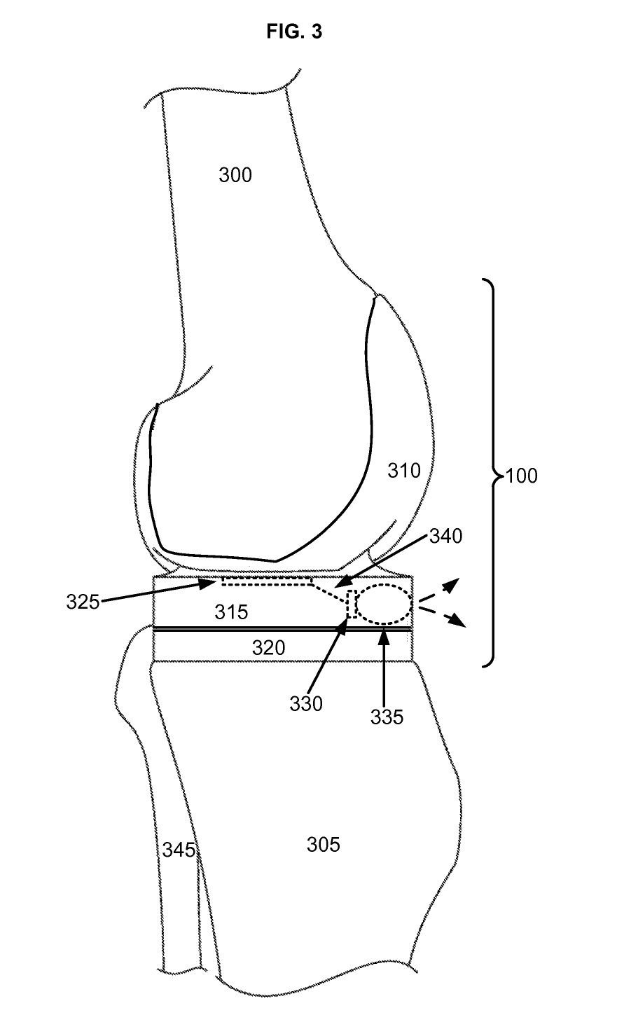

FIG. 3 illustrates aspects of an embodiment of a system for responsive release of a medicament in an artificial joint region. The embodiment shown is an artificial joint 100 that is an artificial knee joint. The artificial joint 100 includes a femoral component 310 attached to the femur 300 as well as a tibial component 320 attached to the tibia 305. The fibula 345 is not directly involved in the artificial joint 100 shown in FIG. 3. The patella is not shown for purposes of illustration, but would be positioned to the right side of the artificial joint 100 shown in FIG. 3. In some embodiments, the patella would be included in the artificial joint. In some embodiments, the patella would not be included in the artificial joint. A tibial spacer 315 is attached to the tibial component 320, the tibial spacer 315 including a load-bearing surface configured to interface with the surface of the femoral component 310 in a usual manner for artificial knee joints.

In the embodiment illustrated in FIG. 3, the tibial spacer 315 includes a system for responsive release of a medicament within the interior of the spacer. The tibial spacer 315 has been fabricated with interior regions conforming to the size and shapes of components of the system for responsive release of a medicament in the artificial joint region. Interior to the tibial spacer 315 is a sensor unit 325. The sensor unit 325 is positioned and configured to sense the loads carried by the artificial knee joint. For example, the sensor unit 325 is positioned to sense the load on the knee generated by the patient walking, running or standing on the artificial knee joint, and configured to sense loads in the expected ranges by a patient engaging in routine activities. The sensor unit is positioned and configured to detect loads on the artificial knee joint. The sensor unit 325 is attached with a wire connector 340 to a responsive release control unit 330. Data from the sensor unit 325 is transmitted along the wire connector 340 to the responsive release control unit 330. For example, in some embodiments the sensor unit 325 includes a piezoresistive element which is configured to send electrical signals along the wire connector 340 in response to loads at the interface of the surfaces of the tibial spacer 315 and the femoral component 310.

The responsive release control unit 330 includes an electronic controller and memory. In some embodiments, the electrical signal generated by one or more piezoresistive elements within the tibial spacer 315 are sufficient to provide adequate electrical power to the responsive release control unit 330 for use by the electronic controller and other components. In some embodiments, the responsive release control unit 330 includes a battery. For example, in some embodiments the responsive release control unit 330 includes a durable battery configured for use over the expected lifetime of the implant (e.g. 10 to 20 years). For example, in some embodiments the responsive release control unit 330 includes a durable battery that can be recharged with electrical signals generated by one or more piezoresistive elements within the tibial spacer 315. In some embodiments, the responsive release control unit includes circuitry configured to quantify the passage of time, e.g. clock cycles in the electric controller. In some embodiments, the responsive release control unit includes circuitry configured to calculate the cumulative load on the artificial knee joint over a period of time, for example one day, one week, or one month. In some embodiments, the responsive release control unit includes circuitry configured to calculate the cumulative load on the artificial knee joint since the surgical implantation of the artificial knee joint. Calculations from the circuitry can be saved in memory, for example to inform later calculations. For example, in some embodiments recent data from one or more load sensors can be compared with a calculation based on historical data from the one or more load sensors, such as average load, average duration of load bearing on the artificial knee joint, historical load high values, and cumulative load on the artificial knee joint since the date of implantation in the patient.

The responsive release control unit 330 is directly attached to an implantable medicament release unit 335 within the tibial spacer 315 in the embodiment shown in FIG. 3. The responsive release control unit 330 is configured to send signals to the attached implantable medicament release unit 335 in response to calculations based on data received from the sensor unit 325. The implantable medicament release unit 335 is configured to send a signal to initiate release of medicament from the reservoir in response to signals accepted from the responsive release control unit 330. The implantable medicament release unit 335 includes a reservoir and a controllable release unit attached to the reservoir. The reservoir is configured and positioned to release medicament stored within the reservoir into the joint region adjacent to the tibial spacer 325. For example, the illustration of FIG. 3 depicts with the dotted arrows the release of a medicament from the reservoir into the joint fluid adjacent to the artificial knee joint. A membrane encircles the artificial knee joint and substantially defines the artificial joint region for the artificial knee. The controllable release unit is positioned and configured to receive signals from the responsive release control unit 330 and to initiate the release of one or more medicaments from the reservoir within the implantable medicament release unit 335 in response to the received signals. For example, one or more medicaments can be released into the joint fluid within the space substantially defined by the membrane.

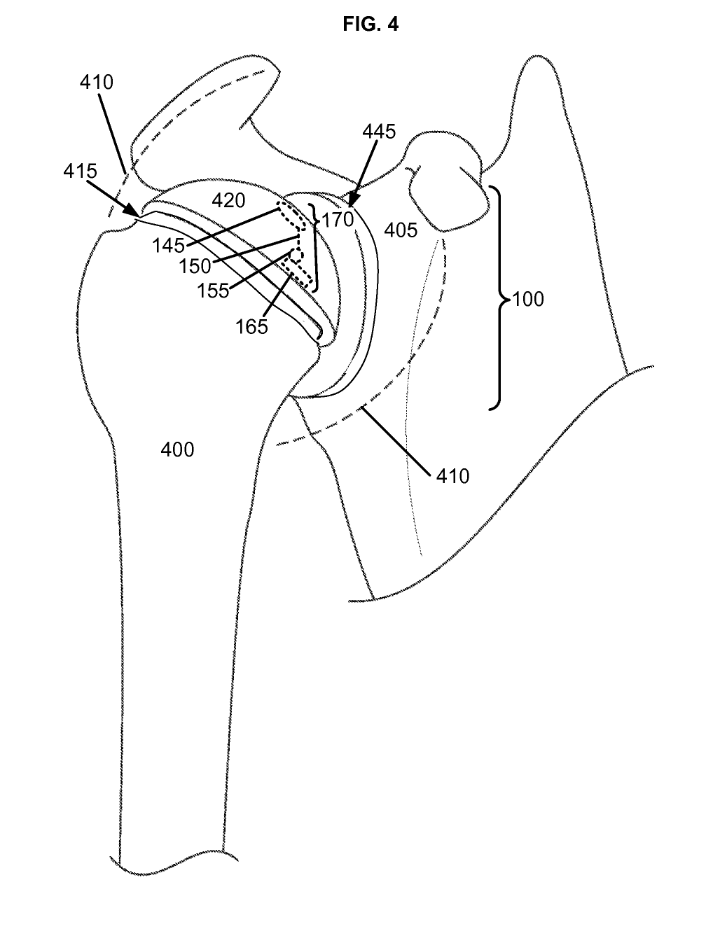

FIG. 4 illustrates an embodiment of an artificial joint 100. The artificial joint 100 shown in FIG. 1 is an artificial shoulder joint. The artificial shoulder joint includes a glenoid component 445 affixed to the glenoid cavity of the scapula bone 405. The artificial shoulder joint includes a humerus spacer 415 attached to the humerus bone 400. A humerus cap 420 is affixed to the humerus spacer 415. A membrane 410 encircles the artificial joint 100 and substantially defines a boundary for the artificial joint region into which medicament can be released. The glenoid component 445, the humerus spacer 415 and the humerus cap 420 can be fabricated from implant-compatible materials, for example implant-compatible metals or plastics.

In the embodiment shown in FIG. 4, the humerus cap 420 includes an internal system for responsive release of a medicament into the artificial shoulder joint region. The humerus cap 420 can be fabricated, in some embodiments, with appropriate internal cavities configured to substantially enclose the components of the internal system. The humerus cap 420 can be fabricated, for example, to surround and substantially enclose the components of the internal system. The internal system for responsive release of a medicament includes an implantable sensor unit 145, including at least one sensor. The implantable sensor unit 145 is positioned and configured to sense load and/or motion within the artificial joint 100. For example, in some embodiments the implantable sensor unit 145 is positioned and configured to sense load within the artificial shoulder joint, for example the load on the humerus cap 420 from the glenoid component 445 during use of the artificial shoulder joint by a patient. For example, in some embodiments the implantable sensor unit 145 is positioned and configured to sense motion within the artificial shoulder joint, for example the motion of the joint relative to the positions of the humerus cap 420 and the glenoid component 445. For example, in some embodiments the implantable sensor unit 145 is positioned and configured to sense both load and motion within the artificial shoulder joint.

In the embodiment shown in FIG. 4, the system for responsive release of a medicament into the artificial shoulder joint region includes a responsive release control unit 155. The responsive release control unit 155 is attached to the implantable sensor unit 145 with a wire connector 150. The implantable sensor unit 145 is configured to transmit signals along the wire connector 150 to the responsive release control unit 155. The responsive release control unit 155 is configured to receive signals from the implantable sensor unit 145 that are transmitted through the wire connector 150. In some embodiments, signals are transmitted from the implantable sensor unit to the responsive release control unit through optical signals transmitted in a fiberoptic carrier. In some embodiments, signals are transmitted from the implantable sensor unit to the responsive release control unit through ultrasound transmissions, and the system may include a wave guide. The responsive release control unit 155 includes an electronic controller and memory.

The responsive release control unit is configured to receive signals sent by the implantable sensor unit, interpret the received signals to provide data regarding the sensed condition(s) (e.g. load and/or motion), and to make calculations based on that data. In some embodiments, the responsive release control unit stores the data into memory. In some embodiments, the responsive release control unit stores the calculations into memory. In some embodiments, the responsive release control unit compares newly-received data to values in one or more look-up tables. For example, a responsive release control unit can be configured to compare newly-received data to values in one or more look-up tables relating to average load expected on an artificial shoulder joint during use by a patient. For example, a responsive release control unit can be configured to compare newly-received data to values in one or more look-up tables relating to average range of motion expected on an artificial shoulder joint during use by a patient. For example, a responsive release control unit can be configured to compare newly-received data to values in one or more look-up tables relating to cumulative load expected on an artificial shoulder joint during use by a patient. For example, a responsive release control unit can be configured to compare newly-received data to values in one or more look-up tables relating to cumulative motion (e.g. in number of movements) expected on an artificial shoulder joint during use by a patient. A responsive release control unit can be configured to calculate values based on newly received data as well as historical data previously saved into memory, for example to calculate values for total load and/or total motion of the artificial shoulder joint based on a combination of newly received data from the sensor unit as well as historical data from memory. A responsive release control unit can be configured to calculate values such as average load over time, average load during a particular time period, average motion over time, or average motion during a particular time period.

A responsive release control unit is configured to send a signal to an implantable medicament release unit in response to data from the sensor unit. In the embodiment shown in FIG. 4, the responsive release control unit 155 is physically attached to the implantable medicament release unit 170, with internal wires transmitting signals from the responsive release control unit 155 to the implantable medicament release unit 170. The implantable medicament release unit 170 includes a reservoir and a controllable release unit attached to the reservoir. The controllable release unit is configured to provide access to the reservoir 165 in response to signals from the responsive release control unit 155. The reservoir 165 includes one or more medicaments for release in response to the controllable release unit.

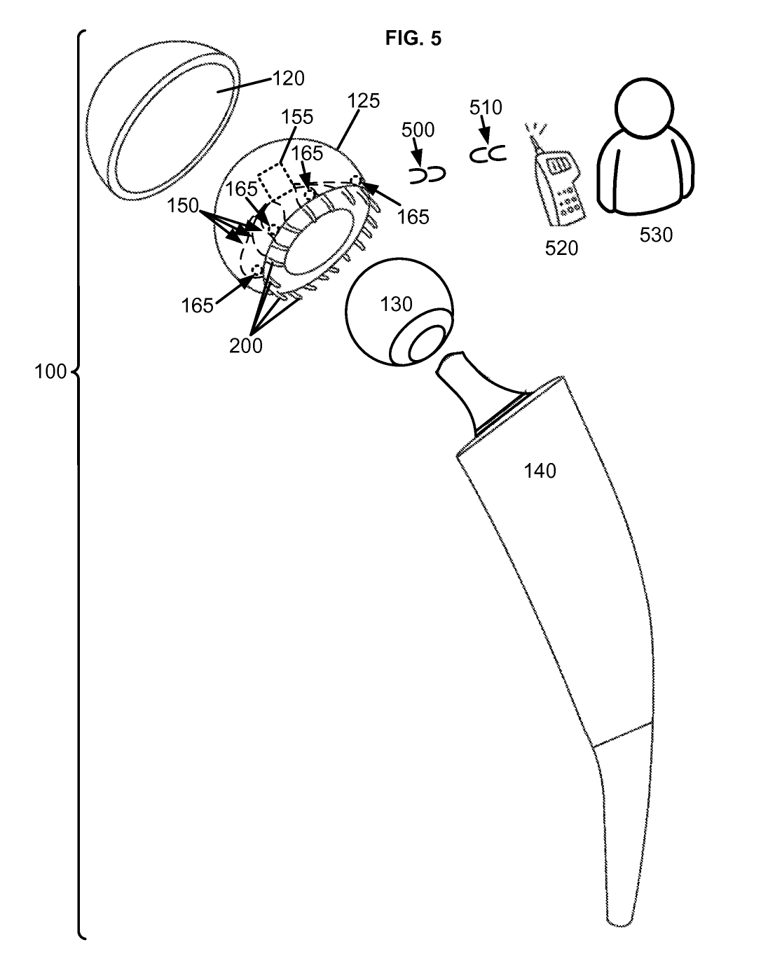

FIG. 5 illustrates aspects of a system for responsive release of a medicament in an artificial joint region. For purposes of illustration, the artificial joint 100 shown in FIG. 5 is depicted outside of a patient, although during use the artificial joint 100 would be implanted in a patient (e.g. as shown in FIGS. 1 and 2). The artificial joint 100 depicted in FIG. 5 is an artificial hip joint. The artificial hip joint includes an acetabular cup 120, configured to be affixed to a corresponding surface of the pelvis of a patient during surgical implantation of the artificial joint 100. The acetabular cup 120 includes an outer surface configured to mate with a corresponding surface of the pelvis of a patient. The acetabular cup 120 includes an inner surface configured to mate with the outer surface of an acetabular liner 125. The artificial hip joint shown in FIG. 5 includes a femoral stem 140 and a femoral ball 130 configured to attach to the terminal end of the femoral stem 140. The exterior surface of the femoral ball 130 is of a size and shape to mate with the inner surface of the acetabular liner 125.

The acetabular liner 125 illustrated in FIG. 5 includes a series of protrusions 200 attached to the edge of the acetabular liner 125 facing the interior of the artificial joint when it is positioned in a patient. The protrusions 200 are attached to a surface of the acetabular liner 125 that is not expected to come into contact with another surface during routine use of the artificial joint 100 by a patient. In the embodiment shown, the protrusions 200 are configured as substantially linear structures. The number, size and position of the protrusions will vary depending on the embodiment. Each of the protrusions 200 includes a sensor positioned and configured to sense the motion of the protrusion during use of the artificial hip joint by a patient. For example, in some embodiments each of the protrusions are flexible (e.g. fabricated from a soft plastic material) and include a piezoelectric element within the protrusion. Each of the piezoelectric elements is positioned and configured to send an electrical signal along an attached wire in response to flexing or bending of the piezoelectric element within the protrusion by motion of the joint and the joint fluid. In some elements, the piezoelectric elements are configured to send an electric signal responsive to the degree of bending or flexing of the protrusion, for example a larger electrical charge in response to a greater degree of flexing relative to a substantially linear protrusion.

In the embodiment shown in FIG. 5, the system for responsive release of a medicament in an artificial joint region includes a responsive release control unit 155 attached to the sensors within the protrusions 200 with wire connectors 150. Each of the sensors within the protrusions 200 is individually connected to the responsive release control unit 155 with a single wire connector 150. Each of the sensors within the protrusions 200 is configured to transmit electrical signals through the attached wire connector 150 to the responsive release control unit 155.

The responsive release control unit includes an electronic controller and memory. In some embodiments, the responsive release control unit includes circuitry. The responsive release control unit is configured to accept transmitted signals from the sensors transmitted through the wire connectors. In some embodiments, the responsive release control unit is configured to calculate motion of the artificial hip joint based on the accepted signals. For example, in some embodiments a responsive release control unit can be configured to calculate motion of the artificial hip joint based on the total number of sensors sending signals. For example, in some embodiments a responsive release control unit can be configured to calculate motion of the artificial hip joint based on the total amount of signal received from the sensors (e.g. the total current generated from all of the sensors). In some embodiments, the responsive release control unit is configured to save the accepted signals, or data calculated from the accepted signals, into memory. In some embodiments, the responsive release control unit is configured to compare accepted signals or data to a historical record from the artificial joint in the patient. In some embodiments, the responsive release control unit is configured to compare accepted signals or data to a set of standard values, for example standard values present in a look-up table.

The responsive release control unit is configured to send signals to one or more of the implantable medicament release units in response to the calculations. For example, in some embodiments a responsive release control unit can be configured to send signals to all of the plurality of implantable medicament release units in response to a calculated value. For example, in some embodiments a responsive release control unit can be configured to send signals to a proportion of the plurality of implantable medicament release units in response to a calculated value. For example, if a calculated value of motion is above a threshold and within a prespecified range, a responsive release control unit can be configured to send signals to half of the plurality of implantable medicament release units, such as those in alternating positions around the edge of the acetabular liner. For example, if a calculated value of motion is above a threshold and within a prespecified range, a responsive release control unit can be configured to send signals to a preselected group of the implantable medicament release units.

In the embodiment shown in FIG. 5, the responsive release control unit 155 includes a transmitter configured to send signals 500 to a remote device 520. For example, in some embodiments the responsive release control unit includes a transmitter in a RFID device. For example, in some embodiments the responsive release control unit includes an ultrasound transmitter. The signals 500 can be received by the remote device 520 and indicated to a user 530. For example, in some embodiments a remote device can be configured to display total motion of the artificial hip joint in the patient, either over the entire lifetime of the artificial joint or for a predetermined time period, in response to receipt of the signals. For example, in some embodiments a remote device can include a cell phone, portable computing device, or a specialized device for monitoring systems for responsive release of a medicament in an artificial joint region. The remote device 520 can be configured to transmit signals 510 to the responsive release control unit 155. The responsive release control unit 155 can include a receiver configured to accept the signals 510 from the remote device 520 and circuitry to determine one or more values from the accepted signals 510. For example, in some embodiments a remote device can send signals indicating a threshold value for cumulative joint motion, and the responsive release control unit is configured to responsively send a signal to the implantable medicament release unit causing the implantable medicament release unit to release one or more medicaments into the joint region.

In some embodiments a remote device can include a cell phone, portable computing device, or a specialized device for monitoring systems for responsive release of a medicament in an artificial joint region. In some embodiments, a remote device can include a receiver. In some embodiments, a remote device can include a receiver and a transmitter. In some embodiments, a remote device is configured to operate at a distance from the patient, such as approximately 5-20 feet away from the skin surface surrounding the artificial joint. In some embodiments, a remote device is configured to operate near to the patient, such as less than approximately 1 foot, or less than approximately 6 inches away from the skin surface surrounding the artificial joint. Although user 530 is shown/described herein as a single illustrated figure, in some embodiments user 530 may be representative of a human user, a robotic user (e.g., computational entity), and/or substantially any combination thereof (e.g., a user may be assisted by one or more robotic agents) unless context dictates otherwise.

In the embodiment shown in FIG. 5, the acetabular cup 125 includes a plurality of implantable medicament release units 170 within the interior of the acetabular cup 125. The implantable medicament release units 170 are positioned along the edge of the acetabular cup 125, adjacent to the surface attached to the proximal ends of the protrusions 200. Each of the implantable medicament release units 170 are attached to the responsive release control unit 155 with a wire connector 150. Each of the implantable medicament release units 170 include a reservoir and a controllable release unit attached to the reservoir. The controllable release unit is configured to provide access to the reservoir in response to signals from the responsive release control unit 155 sent along the corresponding attached wire connector 150. Each of the implantable medicament release units 170 include a reservoir positioned and configured to release one or more medicaments into the joint region, such as into the joint fluid adjacent to the edge of the acetabular cup 125. In some embodiments, each of the implantable medicament release units include a controllable release unit configured to respond to signals accepted from the responsive release control unit. In some embodiments, each of the implantable medicament release units include a reservoir storing the same formulation of medicament(s). In some embodiments, a first subset of the implantable medicament release units include a reservoir configured for storing a first formulation of medicament(s), and a second subset of the implantable medicament release units include a reservoir configured for storing a second formulation of medicament(s).

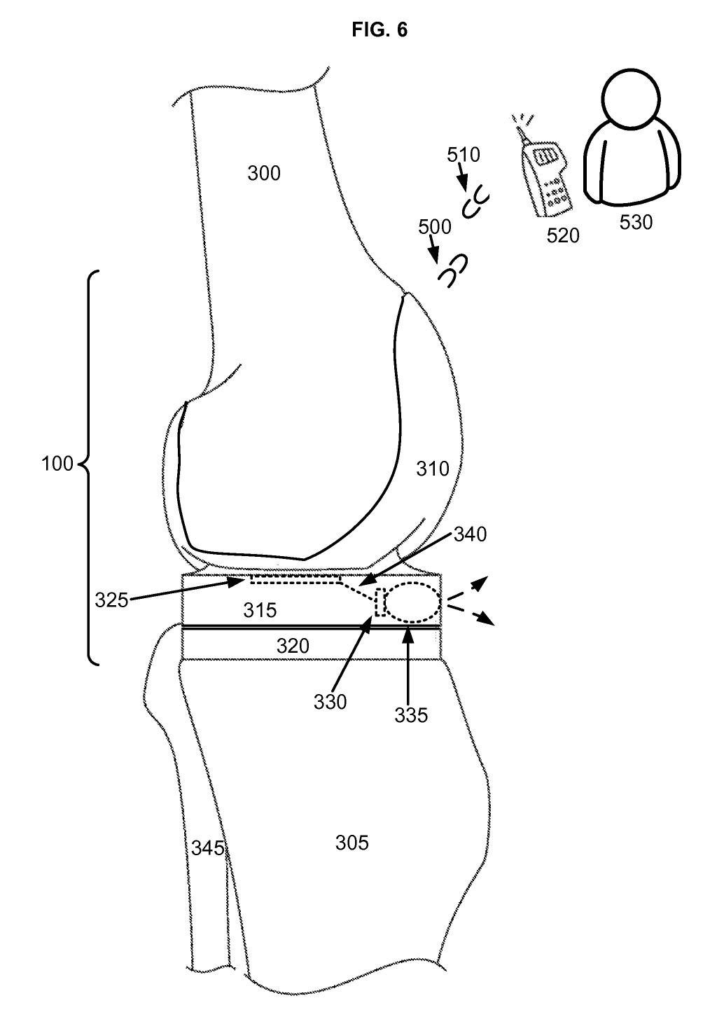

FIG. 6 illustrates aspects of an embodiment of an artificial joint 100 that is an artificial knee joint. The embodiment illustrated in FIG. 6 is similar to the embodiment shown and described in FIG. 3. In the embodiment shown in FIG. 6, the responsive release control unit 330 within the tibial spacer 315 of the artificial knee joint also includes a transmitter and circuitry to control the transmitter. The transmitter is configured to send signals 500 to a remote device 520. The remote device 520 includes a receiver configured to accept the signals 500. The remote device 520 can be operated by a user 530. In the embodiment illustrated in FIG. 6, the remote device 520 includes a transmitter configured to send signals 510 to the responsive release control unit 330.

In some embodiments, a remote device includes an ultrasound receiver and transmitter. In some embodiments, a remote device includes a radio frequency (RF) transmitter and a RF receiver. For example, a remote device can include a transmitter and receiver operational with RFID technology. For example, a remote device can include a transmitter and receiver operating in the UHF spectrum. In some embodiments, the remote device is configured to operate at a distance from the patient, for example the signals can transfer information over a distance of approximately 10 to 20 feet. In some embodiments, the remote device is configured to operate at a position adjacent to the patient, for example the signals can transfer information over a distance of approximately 2 to 6 inches. In some embodiments, the remote device can, for example, display data from the sensor unit to the user. In some embodiments, the remote device can, for example, receive input from a user through a user interface (e.g. a keyboard or a touchscreen) and send instructions in the signals to the artificial joint.

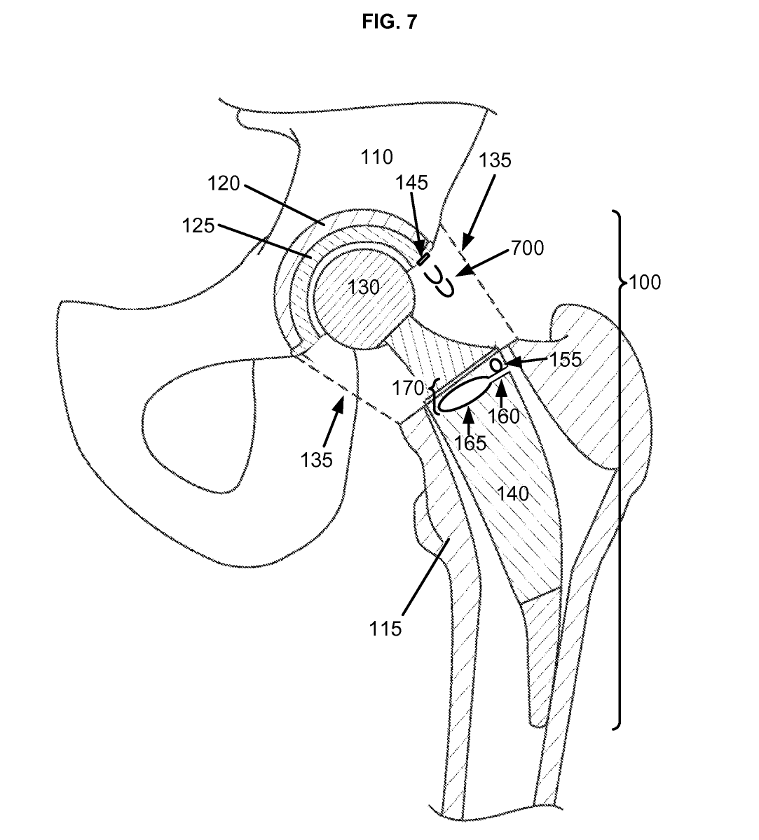

FIG. 7 illustrates aspects of an embodiment of an artificial joint 100 that is an artificial hip joint. The embodiment of the artificial joint 100 shown in FIG. 7 is similar to the embodiment of FIG. 1. In the embodiment shown in FIG. 7, the artificial joint 100 includes an implantable medicament release unit 170 positioned within the femoral shaft 140. The implantable medicament release unit 170 includes a reservoir 165 and a controllable release unit 160 attached to the reservoir 165. A responsive release control unit 155 is affixed to the implantable medicament release unit 170. In the embodiment shown in FIG. 7, the responsive release control unit 155 includes a receiver configured to receive signals 700 from a sensor unit 145 affixed to the acetabular liner 125 of the artificial joint 100. The responsive release control unit 155 includes circuitry configured to process the signals 700 into data relevant to the sensor unit 145. For example, the data can include sensor data, or indicate a lack of data from the sensor unit 145.

The sensor unit 145 shown in FIG. 7 can include a load sensor and/or a motion sensor. The sensor unit 145 is positioned and configured to sense load and/or motion on the artificial joint 100. The sensor unit 145 includes a transmitter configured to send signals 700 to the responsive release control unit 155. The signals can include, for example, data regarding a sensed condition by the sensor unit (e.g. load and/or motion), data indicating a lack of sensed condition by the sensor unit (e.g. the patient has been stationary for a period of time). In some embodiments, the signals are radio frequency (RF) signals. In some embodiments, the signals are ultrasound signals.

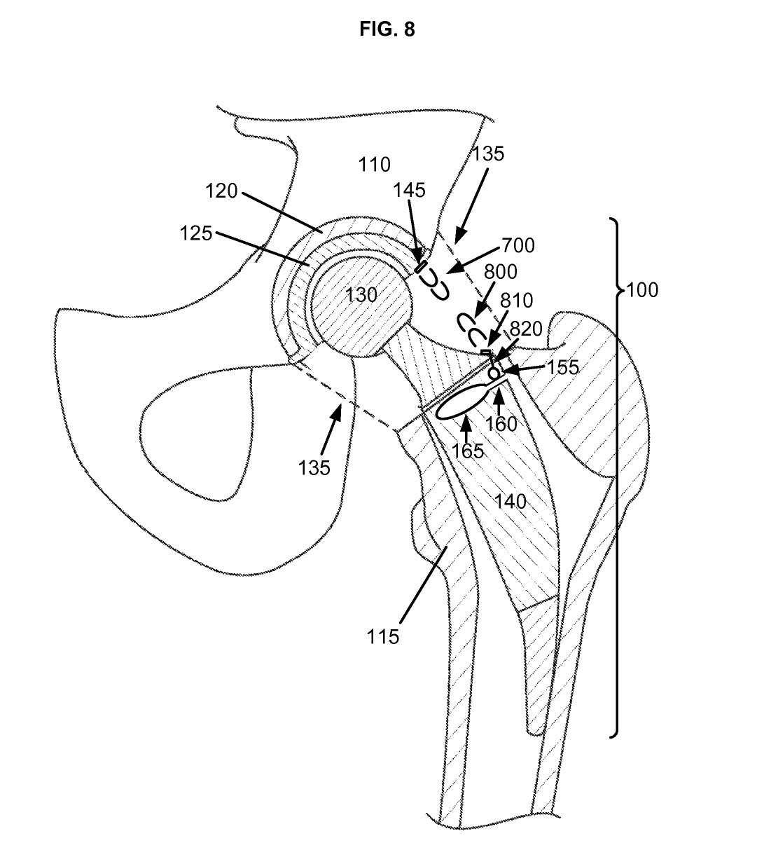

FIG. 8 depicts aspects of an embodiment of an artificial joint 100 that is an artificial hip joint. The embodiment of the artificial joint 100 illustrated in FIG. 8 is similar to the embodiments shown and described in FIGS. 1 and 7. In the embodiment shown in FIG. 8, the artificial joint 100 includes an implantable medicament release unit 170 positioned within the femoral shaft 140. The implantable medicament release unit 170 includes a reservoir 165 and a controllable release unit 160 attached to the reservoir 165.

A responsive release control unit 155 is affixed to the implantable medicament release unit 170. In the embodiment shown in FIG. 8, the responsive release control unit 155 is attached to a transmission unit 810 with a wire connector 820. The transmission unit 810 includes a receiver configured to receive signals 700 from a sensor unit 145 affixed to the acetabular liner 125 of the artificial joint 100. The transmission unit 810 includes a transmitter configured to send signals to the sensor unit 145 affixed to the acetabular liner 125 of the artificial joint 100. The transmission unit 810 includes a transmitter and circuitry configured to send signals through the wire connector 820 to the responsive release control unit 155. The responsive release control unit 155 includes circuitry configured to process signals 700 received by the transmission unit and to process the signals into data relevant to the sensor unit 145. For example, the data can include sensor data, or indicate a lack of data from the sensor unit 145. The responsive release control unit 155 includes circuitry configured to calculate values based on the data, for example maximum and minimum load and/or motion values. The responsive release control unit 155 includes circuitry configured to calculate values based on the data and information stored in memory, for example an average load or motion value based on all of the sensor data from a given time period.

In some embodiments, a system for responsive release of a medicament in an artificial joint includes: at least one fiducial unit configured to attach to a first region of a first component of an artificial joint; an implantable sensor unit configured to attach to a second region of a second component of the artificial joint, the implantable sensor unit including at least one sensor configured to sense a position relative to the at least one fiducial unit; a responsive release control unit including an electronic controller and memory, the responsive release control unit configured to receive signals from the implantable sensor unit and to send signals to an implantable medicament release unit; and the implantable medicament release unit, including at least one reservoir, and at least one controllable release unit configured to respond to signals from the responsive release control unit.

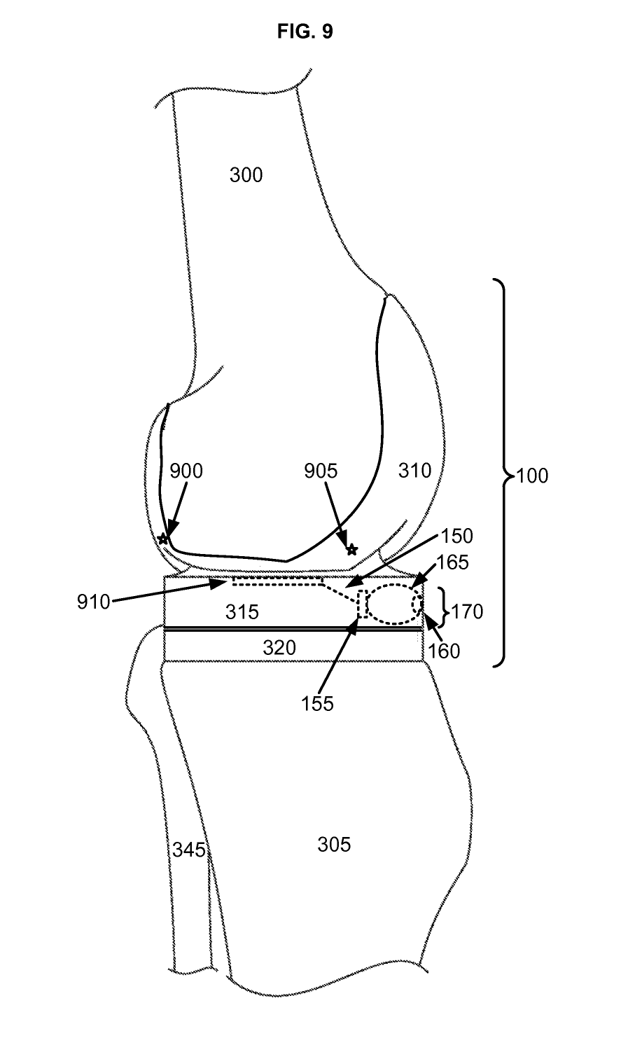

FIG. 9 illustrates aspects of an embodiment of a system for responsive release of a medicament in an artificial joint. FIG. 9 depicts an artificial joint 100 that is an artificial knee joint. The depiction of the artificial joint 100 is similar to those shown and described in FIGS. 3 and 6. In the illustration of FIG. 9, the artificial knee joint includes a tibial component 320 attached to the tibia 305. A tibial spacer 315 is attached to the tibial component 320. The tibial spacer 315 includes a surface configured to reversibly mate with a surface of a femoral component 310 of the artificial joint 100 attached to the femur 300.

In some embodiments, components of the system for responsive release of a medicament in an artificial joint are integrated into the tibial spacer. In the embodiment illustrated in FIG. 9, the tibial spacer 315 includes a portion of a system for responsive release of a medicament in the artificial joint within the interior of the spacer, with the medicament being released into the joint fluid adjacent to the front of the joint. The tibial spacer 315 has been fabricated with interior regions conforming to the size and shapes of components of the system for responsive release of a medicament in the artificial joint region. Interior to the tibial spacer 315 is a sensor unit 910. The sensor unit 910 is positioned and configured to sense the position of the sensor unit 910 relative to one or more fiducial units 900, 905. In some embodiments, a sensor unit is positioned and configured to sense a load on the artificial joint. For example, the sensor unit 910 illustrated in the embodiment of FIG. 9 can detect the mass pressure at the interface between a surface of the femoral component 310 and a surface of the tibial spacer 315.

In some embodiments, a sensor unit is positioned and configured to sense loads carried by the artificial knee joint. For example, the sensor unit 910 shown in FIG. 9 is positioned to sense the load on the knee generated by the patient walking, running or standing on the artificial knee joint, and configured to sense loads in the expected ranges by a patient engaging in routine activities. The sensor unit is positioned and configured to detect loads on the artificial knee joint. The sensor unit 910 is attached with a wire connector 150 to a responsive release control unit 155. Data from the sensor unit 910 is transmitted along the wire connector 150 to the responsive release control unit 150. For example, in some embodiments the sensor unit 910 includes a piezoresistive element which is configured to send electrical signals along the wire connector 150 in response to loads at the interface of the surfaces of the tibial spacer 315 and the femoral component 310.

Some embodiments include at least one fiducial unit. A “fiducial unit,” as used herein, includes a unit used as a standard of reference for measurement within the artificial joint. In some embodiments, one or more fiducial units are affixed in a location within the artificial joint. In some embodiments, a fiducial unit is a physical unit affixed to a surface of a component of the artificial joint. In some embodiments, a fiducial unit is a physical unit integral to a component of the artificial joint. A fiducial unit is configured and fabricated to be detectable by a sensor unit within the system for responsive release of a medicament in an artificial joint. Some embodiments include a single fiducial unit and a sensor unit. Some embodiments include a plurality of fiducial units and a sensor unit. For example, some embodiments include a fiducial unit that is fabricated to include one or more ferromagnetic materials, and a sensor unit that includes a magnetic detector configured to detect the relative strength of the magnetic field depending on the distance between the sensor unit and the fiducial unit at a time point. For example, some embodiments include a fiducial unit that is fabricated to include a surface reflective to one or more energy beams, and a sensor unit including a transmitter and a receiver of the one or more energy beams. For example, some embodiments include a fiducial unit that includes a surface positioned and configured to reflect ultrasound energy beams, and a sensor unit including a ultrasound transmitter and receiver. The sensor unit is configured to detect the relative position of the fiducial unit based on the reflected ultrasound beams returning to the sensor unit, such as the reflective angle, strength and scatter of the reflected ultrasound beams. See Stoll et al., “Passive Markers for Tracking Surgical Instruments in Real-Time 3-D Ultrasound Imaging,” IEEE Transactions on Medical Imaging, 31(3): 563-575 (2012), which is incorporated by reference. In some embodiments, a fiducial unit can include a durable contrast agent. See, for example, Delogu et al., “Functionalized Multiwalled Carbon Nanotubes as Ultrasound Contrast Agents,” PNAS 109(41): 16612-16617 (2012), which is incorporated by reference. For example, some embodiments include a fiducial unit that includes a surface positioned and configured to reflect RF energy beams, and a sensor unit including an RF transmitter and receiver. The sensor unit is configured to detect the relative position of the fiducial unit based on the reflected RF beams returning to the sensor unit, such as the reflective angle, strength and scatter of the reflected RF beams.

In some embodiments, a fiducial unit includes at least one encapsulated material. For example, a fiducial unit can include a ferromagnetic material encapsulated within a bio-compatible plastic material. For example, a fiducial unit can include a metal reflective to RF waves that is encapsulated within a bio-compatible plastic material. Some embodiments include at least one fiducial unit configured to be a substantially planar structure. For example, at least one fiducial unit can be positioned and configured to form a surface reflective to ultrasound waves. For example, at least one fiducial unit can be positioned and configured to form a smooth surface at the interface region between two components of an artificial joint, so as to permit smooth motion of the joint. In some embodiments, a fiducial unit is configured to be affixed to a first region of a first component of the artificial joint. In some embodiments, a fiducial unit is configured to be affixed to a second region of a second component of the artificial joint. In some embodiments, a fiducial unit is integral to a first region of a first component of the artificial joint. In some embodiments, a fiducial unit is integral to a second region of a second component of the artificial joint. Some embodiments include: a plurality of fiducial units, each fiducial unit configured to attach to the first region of the first component of the artificial joint; and wherein the implantable sensor unit includes at least one sensor configured to sense the position of each of the plurality of fiducial units.

In the embodiment illustrated in FIG. 9, the sensor unit 910 is positioned within the tibial spacer 315, at a location adjacent to the surface of the tibial spacer 315 that mates with the surface of the femoral component 310 of the artificial joint 100. A first fiducial unit 900 and a second fiducial unit 905 are affixed to the femoral component 310. In the embodiment illustrated, the first fiducial unit 900 is affixed to the femoral component 310 at a location posterior to the sensor unit 910 relative to the position of the artificial joint 100 in use in a patient. The second fiducial unit 905 is affixed to the femoral component 310 at a location anterior to the sensor unit 910 relative to the position of the artificial joint 100 in use in a patient. Some embodiments can include a third fiducial unit, a fourth fiducial unit, and/or subsequently-numbered fiducial units (e.g. fifth fiducial unit, sixth fiducial unit, etc.) as required by the embodiment. For example, a larger joint may require a larger number of fiducial units for a sensor unit to detect the full range of motion of the joint. For example, a joint with an extended range of motion may require more fiducial units than a joint of a type that is expected to have a smaller range of motion. The type, number, and position of fiducial units in an embodiment will depend on factors including the type of artificial joint, the fabrication material of the artificial joint, the expected range of use and/or duration of use of the artificial joint. Some embodiments include fiducial units of different sizes and/or shapes as needed to be detected by a sensor unit. Fiducial units can be positioned at the edges of the artificial joint, or at the edges of a weight-bearing surface of an artificial joint, as needed for reference by a sensor unit to detect the motion of an artificial joint.

In some embodiments, a sensor unit includes a motion sensor. In some embodiments, a sensor unit includes a load sensor. In some embodiments, a sensor unit includes both a sensor unit and a load sensor. In some embodiments, a sensor unit includes a distance sensor, configured to detect a distance relative to the at least one fiducial unit. In some embodiments, a sensor unit includes a transmitter. A sensor unit can be an implantable sensor unit.

During movement of the joint, a sensor unit can detect the relative distance and/or position between one or more fiducial units and the sensor unit. This data is transmitted to a responsive release control unit. For example, in the embodiment shown in FIG. 9, data from the sensor unit 910 is transmitted to the responsive release control unit 155 through a wire connector 150. The sensor unit 910 includes a transmitter unit configured to send data from the sensor unit, for example load data from detected loads, and/or joint motion data, to the responsive release control unit 155 through the wire connector 150. The responsive release control unit 155 is directly connected to an implantable medicament release unit 170. The implantable medicament release unit 170 includes a reservoir 165 and a controllable release unit 160 attached to the reservoir 165. The controllable release unit 160 illustrated in FIG. 9 is physically positioned between the reservoir 165 and a joint fluid space adjacent to the tibial spacer 315 so as to control the release of at least one medicament into the joint fluid adjacent to the tibial spacer 315 (i.e. to the right of the illustration in FIG. 9).

In some embodiments, an implantable medicament release unit is modular. For example, an implantable medicament release unit can be fabricated with modules that can be changed or swapped with other components during fabrication of the system. In some embodiments, the controllable release unit of the implantable medicament release unit includes one or more electrical pulse generators. For example, a controllable release unit can include one or more electrical pulse generators configured and positioned to initiate release of at least one medicament from a reservoir in response to an electrical pulse. In some embodiments, the controllable release unit of the implantable medicament release unit includes one or more optical pulse generators. For example, a controllable release unit can include one or more optical pulse generators configured and positioned to initiate release of at least one medicament from a reservoir in response to an optical pulse. In some embodiments, the controllable release unit of the implantable medicament release unit includes one or more devices positioned to reversibly produce physical pressure on the reservoir. For example, a controllable release unit can include one or more flanges, valves or clamps configured and positioned to reversibly produce physical pressure on the reservoir, and thereby reversibly initiate release of at least one medicament from the reservoir.

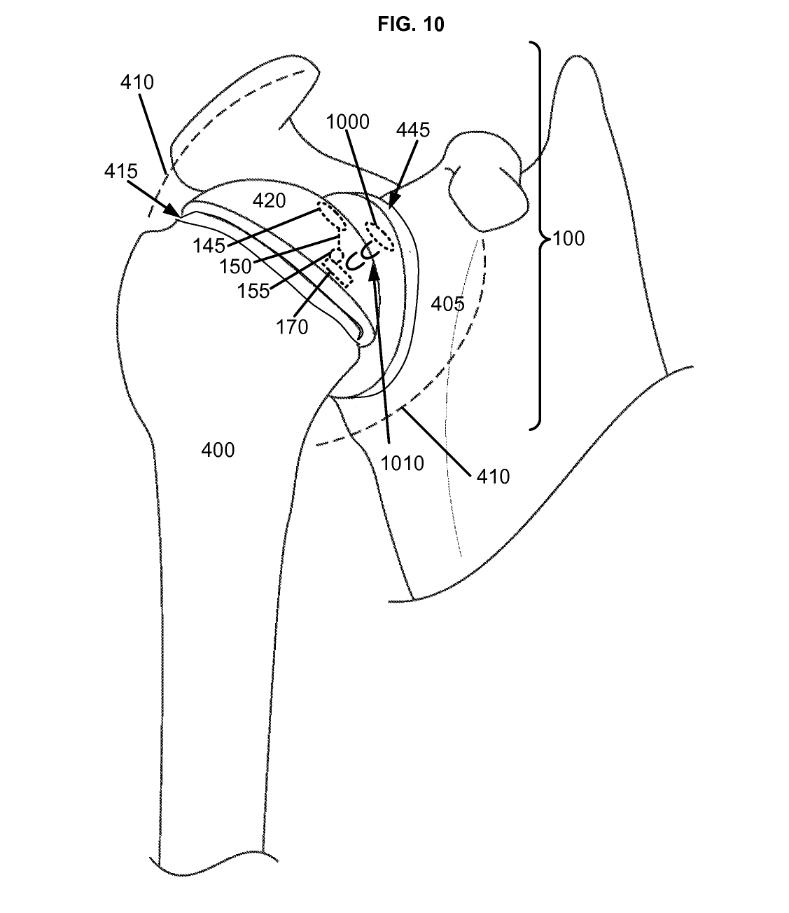

FIG. 10 illustrates an embodiment of a system for responsive release of a medicament in an artificial joint. The artificial joint 100 shown in FIG. 10 is an artificial shoulder joint, similar to that shown in FIG. 4. The artificial shoulder joint includes a glenoid component 445 affixed to the glenoid cavity of the scapula bone 405. The artificial shoulder joint includes a humerus spacer 415 attached to the humerus bone 400. A humerus cap 420 is affixed to the humerus spacer 415. A membrane 410 encircles the artificial joint 100 and is a boundary for the artificial joint region into which medicament can be released. For example, at least one medicament can be released from the system for responsive release of a medicament into the joint fluid within the boundary of the membrane 410 in vivo.

The artificial joint 100 illustrated in FIG. 10 includes an implantable sensor unit 145 attached with a wire connector 150 to a responsive release control unit 155. The sensor unit 145 is positioned and configured to detect load on the joint 100, such as force against the sensor unit 145 at a vector from the surface of the glenoid component 445. The system for responsive release of a medicament shown in FIG. 10 includes an implantable medicament release unit 170. The implantable medicament release unit 170 includes a reservoir and a controllable release unit attached to the reservoir. The implantable medicament release unit 170 is positioned and configured to release at least one medicament from the reservoir into the joint fluid adjacent to the artificial joint 100 within the boundary defined by the membrane 410. The implantable medicament release unit 170 includes a receiver configured to receive signals 1010 from a fiducial unit 1000. A fiducial unit 1000 is integral to the glenoid component 445. In some embodiments, a fiducial unit transmits signals, for example magnetic waves from a ferromagnetic material within the fiducial unit. In some embodiments, a fiducial unit reflects signals that can be detected by the implantable medicament release unit. For example, a fiducial unit can be configured and positioned to reflect RF waves that are signals that can be detected by the implantable medicament release unit.

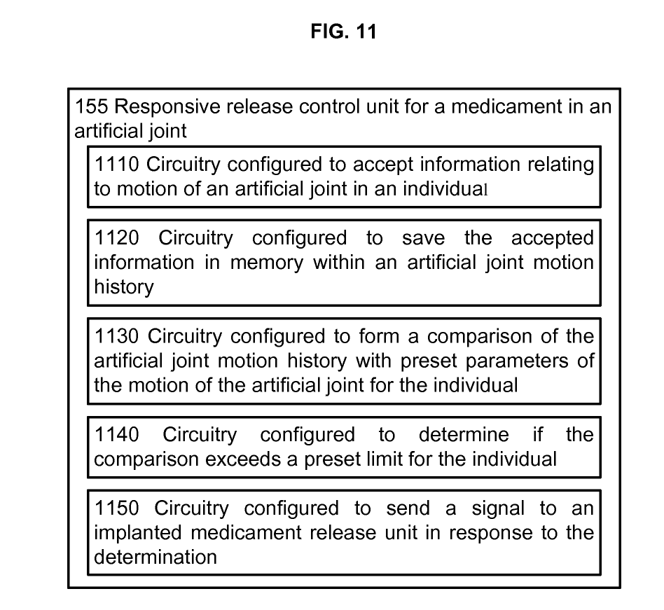

Some embodiments include a responsive release control unit for a medicament in an artificial joint including circuitry. FIG. 11 depicts a responsive release control unit for a medicament in an artificial joint 155. The responsive release control unit for a medicament in an artificial joint 155 includes: circuitry configured to accept information relating to motion of an artificial joint in an individual 1110; circuitry configured to save the accepted information in memory within an artificial joint motion history 1120; circuitry configured to form a comparison of the artificial joint motion history with preset parameters of the motion of the artificial joint for the individual 1130; circuitry configured to determine if the comparison exceeds a preset limit for the individual 1140; and circuitry configured to send a signal to an implanted medicament release unit in response to the determination 1150.

As shown in FIG. 11, a responsive release control unit for a medicament in an artificial joint 155 includes: circuitry configured to accept information relating to motion of an artificial joint in an individual 1110. In some embodiments the circuitry configured to accept information relating to motion of an artificial joint in an individual includes a receiver. For example, the circuitry can include a RF receiver. For example, the circuitry can include an ultrasound receiver. For example, the circuitry can include an electrical signal receiver. In some embodiments the circuitry configured to accept information relating to motion of an artificial joint in an individual includes circuitry configured to accept information regarding a load on the artificial joint during motion. For example, the circuitry can be configured to accept information regarding load during a time period, such as an hour, a day or a week. For example, the circuitry can be configured to accept information regarding a maximum load. For example, the circuitry can be configured to accept information regarding a minimum load. In some embodiments the circuitry configured to accept information relating to motion of an artificial joint in an individual includes circuitry configured to accept information regarding repetition of motion of the artificial joint. For example, the circuitry can be configured to accept information regarding the number of repetitions of use of the joint during a time period, such as an hour, a day or a week. In some embodiments the circuitry configured to accept information relating to motion of an artificial joint in an individual includes circuitry configured to accept information regarding extension of the artificial joint. For example, the circuitry can be configured to accept information regarding a maximum extension of the joint during a time period, such as an hour, a day or a week.

The responsive release control unit for a medicament in an artificial joint 155 includes: circuitry configured to save the accepted information in memory within an artificial joint motion history 1120. In some embodiments the circuitry configured to save the accepted information in memory within an artificial joint motion history includes non-volatile memory. In some embodiments the circuitry configured to save the accepted information in memory within an artificial joint motion history includes volatile memory. In some embodiments the circuitry configured to save the accepted information in memory within an artificial joint motion history includes RAM memory.

FIG. 11 illustrates that a responsive release control unit for a medicament in an artificial joint 155 includes circuitry configured to form a comparison of the artificial joint motion history with preset parameters of the motion of the artificial joint for the individual 1130. In some embodiments, the circuitry configured to form a comparison of the artificial joint motion history with preset parameters of the motion of the artificial joint for the individual includes circuitry configured to compare information regarding a number of movements of the artificial joint. For example, the circuitry can be configured to compare information regarding a number of movements of the artificial joint during a time period with an average for that time period. In some embodiments, the circuitry configured to form a comparison of the artificial joint motion history with preset parameters of the motion of the artificial joint for the individual includes circuitry configured to compare information regarding extension of the artificial joint. For example, information regarding extension can include expected parameters of extension, such as average, expected maximum, or expected minimum, for that model of artificial joint. For example, the circuitry can be configured to compare information regarding extension of the artificial joint with a prior maximum extension of the joint during a time period, such as an hour, a day or a week. In some embodiments, the circuitry configured to form a comparison of the artificial joint motion history with preset parameters of the motion of the artificial joint for the individual includes circuitry configured to compare information regarding a cumulative load on the artificial joint. For example, information regarding a cumulative load on the artificial joint can include a maximum lifetime expected load for that model of artificial joint, or a historical cumulative load for that particular joint in a patient.

FIG. 11 shows that a responsive release control unit for a medicament in an artificial joint 155 includes circuitry configured to determine if the comparison exceeds a preset limit for the individual 1140. In some embodiments, circuitry configured to determine if the comparison exceeds a preset limit for the individual includes circuitry operating a look-up table. For example, the look-up table can include maximum load values for that model of artificial joint. For example, the look-up table can include maximum extension values for that model of artificial joint. In some embodiments, circuitry configured to determine if the comparison exceeds a preset limit for the individual includes circuitry operating stored memory. In some embodiments, circuitry configured to determine if the comparison exceeds a preset limit for the individual includes circuitry configured to send a wireless signal. In some embodiments, circuitry configured to determine if the comparison exceeds a preset limit for the individual includes circuitry configured to send a signal through a wire connection. For example, the circuitry can be operable to initiate a signal from an attached transmitter. In some embodiments, circuitry configured to determine if the comparison exceeds a preset limit for the individual includes circuitry configured to send a release signal. For example, the circuitry can be operable to initiate a release signal for release of a medicament from the reservoir.

In some embodiments, a responsive release control unit for a medicament in an artificial joint, such as shown in FIG. 11, also includes circuitry configured to send a signal to a user interface in response to the determination. For example, the circuitry can include circuitry configured to send a signal from an attached transmitter to a user interface of a remote device external to the artificial joint. In some embodiments, a responsive release control unit is configured to be implantable. For example a responsive release control unit can be included in an implantable system with an artificial joint. In some embodiments, a responsive release control unit is configured to be handheld. For example, in some embodiments a responsive release control unit is included in a handheld remote device distinct from the artificial joint.

Although the embodiments and examples herein are described relative to human patients and human joints, in some embodiments a system for responsive release of a medicament in an artificial joint region can be implanted into a non-human animal and utilized within the animal. A patient, as used herein, can include a human patient. A patient, as used herein, can include a non-human patient. For example, in some embodiments a system for responsive release of a medicament in an artificial joint region can be implanted into a patient that is one of: a canine, a feline, a bovine, or a swine.

The state of the art has progressed to the point where there is little distinction left between hardware, software (e.g., a high-level computer program serving as a hardware specification), and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software (e.g., a high-level computer program serving as a hardware specification), and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software (e.g., a high-level computer program serving as a hardware specification) implementation; or, yet again alternatively, the implementer may opt for some combination of hardware, software (e.g., a high-level computer program serving as a hardware specification), and/or firmware in one or more machines, compositions of matter, and articles of manufacture, limited to patentable subject matter under 35 U.S.C. §101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Optical aspects of implementations will typically employ optically-oriented hardware, software (e.g., a high-level computer program serving as a hardware specification), and or firmware.

In some implementations described herein, logic and similar implementations may include computer programs or other control structures. Electronic circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software (e.g., a high-level computer program serving as a hardware specification) or firmware, or of gate arrays or programmable hardware, such as by performing a reception of or a transmission of one or more instructions in relation to one or more operations described herein. Alternatively or additionally, in some variants, an implementation may include special-purpose hardware, software (e.g., a high-level computer program serving as a hardware specification), firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

In a general sense, the various aspects described herein can be implemented, individually and/or collectively, by a wide range of hardware, software (e.g., a high-level computer program serving as a hardware specification), firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein may be implemented in an analog or digital fashion or some combination thereof.

Alternatively or additionally, implementations may include executing a special-purpose instruction sequence or invoking circuitry for enabling, triggering, coordinating, requesting, or otherwise causing one or more occurrences of virtually any functional operation described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). It will be possible to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood that each function and/or operation within such block diagrams, flowcharts, or examples can be implemented, individually and/or collectively, by a wide range of hardware, software (e.g., a high-level computer program serving as a hardware specification), firmware, or virtually any combination thereof, limited to patentable subject matter. In an embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, limited to patentable subject matter, and that designing the circuitry and/or writing the code for the software (e.g., a high-level computer program serving as a hardware specification) and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In some instances, one or more components may be referred to herein as “configured to,” “configured by,” “configurable to,” “operable/operative to,” “adapted/adaptable,” “able to,” “conformable/conformed to,” etc. Such terms (e.g. “configured to”) generally encompass active-state components and/or inactive-state components and/or standby-state components, unless context requires otherwise.

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

The herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

### PROPHETIC EXAMPLES

### Example 1

### Responsive Drug Delivery System for an Artificial Hip Joint

An implanted artificial hip joint is subject to osteolysis and bone resorption which may cause loosening of the implanted joint and require additional surgery to reset the prosthesis (see e.g., Agarwal, “Osteolysis—Basic Science, Incidence and Diagnosis,” Current Orthopaedics 18:220-231 (2004) and Collier et al., “Osteolysis After Total Knee Arthroplasty: Influence of Tibial Baseplate Surface Finish and Sterilization of Polyethylene Insert” J. Bone Joint Surg. 87-A: 2702-2708, (2005), which are each incorporated herein by reference). A responsive drug delivery system is incorporated in the artificial hip joint to monitor motion and load forces on the joint and deliver anti-osteolytics and bone growth factors when necessary. The system includes: sensor units to monitor the artificial joint, a controller to process data from the sensors and a drug release unit which responds to signals from the controller to release osteogenic and anti-osteolytic drugs.

The hip joint prosthesis has a femoral component which includes a head (or ball), a neck attached to the head and a stem which is implanted in the medullary canal. There is also an acetabular component which forms a socket that includes an outer and inner cup with the outer cup attached to the pelvic bone and the inner cup bearing the head of the femoral component. The neck and stem of the femoral component are fabricated from titanium. See e.g., U.S. Pat. No. 6,761,741, “Prosthetic Joint” issued to Iesaka on Jul. 13, 2004 which is incorporated herein by reference. For example, a femoral component with a titanium stem and a cobalt chromium alloy head is available from Stryker Orthopaedics, Mahwah, N.J.

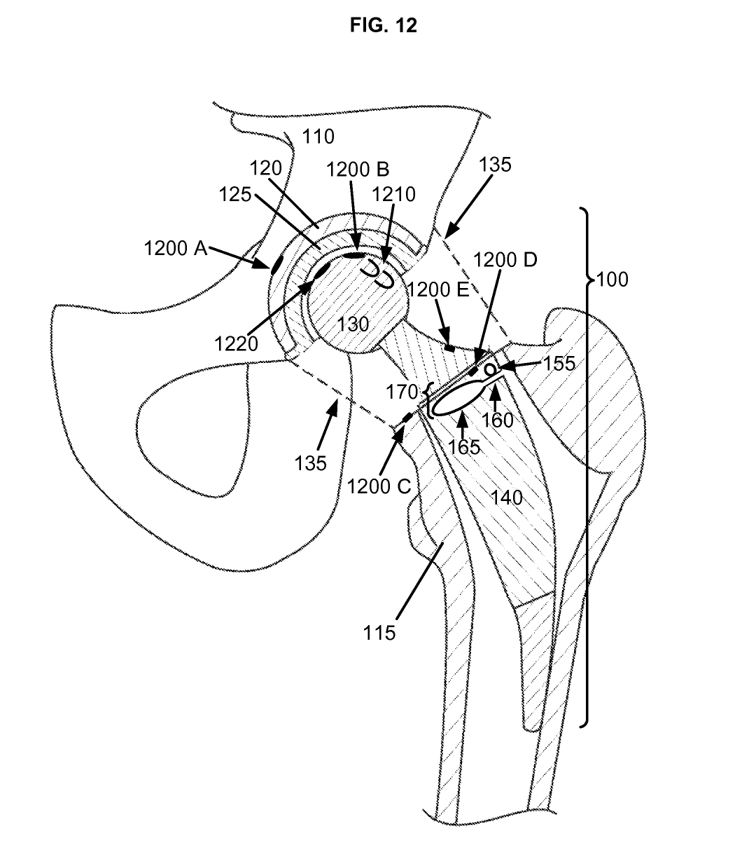

The artificial hip joint incorporates sensor units to detect the position, alignment and motion of the artificial hip joint. Sensor units including pulse echo A-mode ultrasound transducers are used to localize selected surface regions on the artificial joint. For example, as illustrated in FIG. 12, immersion unfocused 3.5 MHz ultrasound transducers in sensor units 1200 A, 1200 B, 1200 C, 1200 D, 1200 E (available from Olympus Corp., Waltham, Mass.) are attached to the hip joint prosthesis on the acetabular component 1200 A, and the femoral component 1200 B, 1200 D, 1200 E (e.g., the head, the neck, the stem), and to the proximal femur bone 1200 C. The plurality of sensor units including ultrasound transducers 1200 A, 1200 B, 1200 C, 1200 D, 1200 E are collectively referred to as “ultrasound sensors” with reference to FIG. 12. One or more ultrasound sensors, accelerometers, load sensors, and associated components are integrated into a “sensor unit” for placement at a location on the artificial hip joint.

The ultrasound sensors detect and localize the surfaces of the hip joint prosthesis and associated bones and body tissues. In addition, the prosthesis is constructed with accelerometers in each sensor unit. The accelerometers track the position of each ultrasound sensor, and the motion of the artificial hip joint. The use of ultrasound sensors and accelerometers to localize and track the motion of bone joints and surrounding ligaments and soft tissue are described (see e.g., U.S. Patent Application No. 2010/0198067 “Noninvasive Diagnostic System” by Mahfouz et al. published Aug. 5, 2010 and U.S. Pat. No. 5,533,519 “Method and Apparatus for Diagnosing Joints” issued to Radke et al. on Jul. 9, 1996 which are incorporated herein by reference). The ultrasound sensors and the accelerometers transmit wireless signals to a control unit (e.g., control unit with a controller, circuitry, memory and microprocessors) which processes and retains the accumulated data. For example, FIG. 12 depicts signals 1210 transmitted from sensor unit 1200 B to the responsive release control unit 155.

Load sensors are also incorporated in the artificial hip joint to monitor cumulative load forces placed on the prosthesis. For example, load sensors based on piezoelectric cantilevers may be placed on the outer acetabular cup and on the femoral head of the prosthesis (see e.g., U.S. Patent Application No. 2011/0124981 “Method for Detecting Body Parameters” by Roche published May 26, 2011 which is incorporated herein by reference). Microcantilever sensors for detecting loads applied to orthopedic joints are described (see e.g., U.S. Pat. No. 7,097,662 “In-Vivo Orthopedic Implant Diagnostic Device for Sensing Load, Wear and Infection” issued to Evans, III et al. on Aug. 29, 2006 which is incorporated herein by reference). Microscale cantilever sensors with transmitters communicate the cumulative load forces applied to the hip joint and the signals are processed along with the motion data. The raw signal data and the processed information are stored in memory in the control unit. For example, FIG. 12 depicts sensor unit 1220, including a load sensor, and positioned to detect load on the ball 130 during use of the artificial joint 100.

Cumulative motion cycles of the hip joint, for example, the number of times the femur rotates ≧15 degrees (as in walking, or jogging) are tabulated and compared to predetermined limits for the specific hip joint prosthesis. Cumulative load forces, for example, the axial load on the hip joint in Newtons, are integrated over time to calculate the cumulative load for the individual using the artificial joint during the monitored time period. The cumulative motion and load data are compared to predetermined parameters for the prosthetic device which may be based on clinical data or laboratory simulations with the prosthesis. If motion or load parameters exceed preset limits, then the controller signals a drug delivery unit to deliver anti-osteolytic and/or osteogenic agents. The cumulative load, the cumulative motion or the sum of the cumulative load and motion parameters may be compared to preset values for the specific prosthetic hip joint. Signaling by the controller activates the drug delivery unit to deliver anti-osteolytic agents and/or bone growth factors to specific locations in the artificial joint.

Drug delivery units incorporated in the artificial hip joint respond to the controller by delivering anti-osteolytic agents and bone growth factors to the interface between the artificial hip joint components and the patient's bones. The drug delivery unit may have a reservoir with an outlet, a valve and pump to deliver anti-osteolytic drugs to multiple locations within the hip joint. For example, drugs may be delivered to the femoral stem of the artificial joint at the interface with the medullary canal of the patient's proximal femur, and to the interface between the outer acetabular cup and the patient's hip bone. The drug delivery unit includes a medicament applicator comprised of hollow microneedle arrays that are connected to a reservoir containing medicaments. Hollow microneedle arrays may be fabricated using microfabrication technology adapted from the microelectronics industry. For example silicon hollow microneedle arrays may be fabricated by etching holes through silicon wafers using deep reactive ion etching and then etching microneedles around the holes. See, e.g., McAllister et al., “Microfabricated Needles for Transdermal Delivery of Macromolecules and Nanoparticles: Fabrication Methods and Transport Studies,” Proc. Natl. Acad. Sci. USA, 100: 13757-13760, (2003) which is incorporated herein by reference. Microneedle arrays (10×10) containing 100 microneedles in an area of 3×3 mm are constructed with conical microneedles approximately 150 μm in length; with a base diameter of approximately 80 μm and a tip with approximately a 1 μm radius of curvature. Hollow microneedles with diameters between 35 μm and 300 μm and lengths between 150 μm and 1000 μm may be fabricated as shown by McAllister et al., Ibid. Alternatively hollow microneedles may be fabricated from metals (e.g., Ni or NiFe) or polymers (e.g., polyglycolic acid and poly lactic acid) by using micromolds or by electroplating polymer microneedles with nickel as shown by McAllister et al., Ibid. Hollow microneedle arrays may be connected via a manifold to a mini-pump, solenoid valve actuators and to a reservoir containing medicaments. Mini-pumps and solenoid valves are available from Parker-Hannifin, Precision Fluidics Division, Hollis, N.H. The medicament applicator, comprised of hollow microneedle arrays, solenoid valve actuators, a minipump and a reservoir is embedded in the prosthetic device with the hollow microneedles facing the proximal femur and hip bone interfaces with the prosthesis. Multiple medicament applicator units may be embedded in the hip prosthesis to deliver drugs to different regions of the hip joint (e.g., anterior, posterior, lateral, medial, superior, inferior).

The drug delivery unit with a medicament applicator has a power source and micro-circuitry to allow responsive delivery of the specific drug (e.g., anti-osteolytic or osteogenic) at the required dose and schedule. An integrated lithium battery can provide electric current to drive solenoid actuator valves and a minipump which are connected to the medicament reservoir and microneedle arrays. The controller, responding to data from the motion and load sensors, activate drug delivery and monitors the total dose of drug delivered and retains the information to prevent exceeding the maximum recommended dose and to inform the doctor and patient. The micro-circuitry can record and store total dosage and/or dosage within a fixed time period of hours, days, weeks or months. The controller may transmit information including drug dosage, schedule, and drug consumption to a computer network system that includes the patient, the patient's family, healthcare providers, insurance companies, regulatory authorities and public health officials.

### Example 2

### Responsive Drug Delivery System for an Artificial Knee Joint

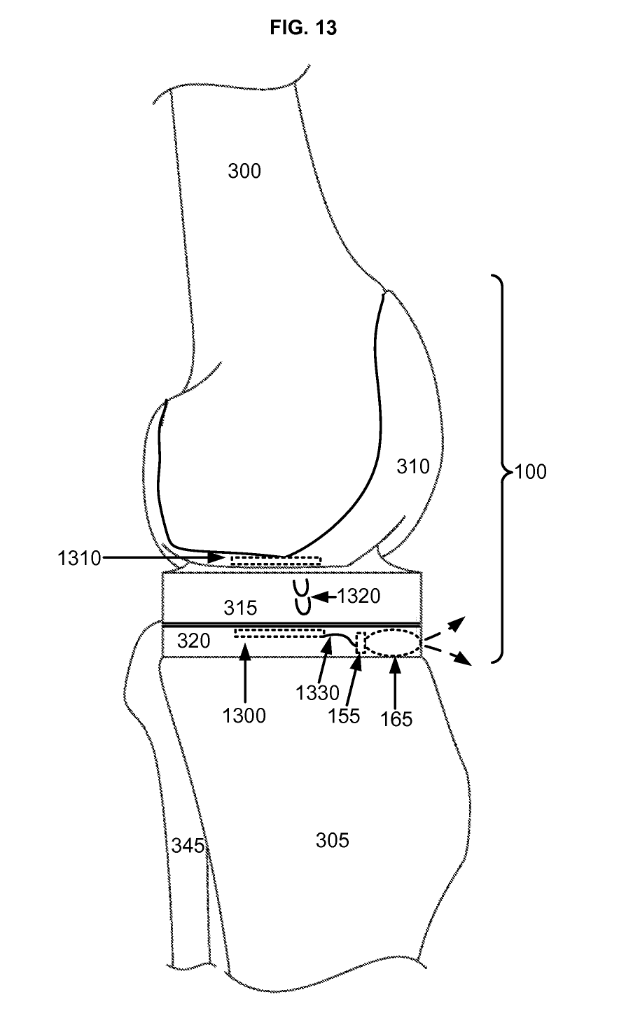

A responsive drug delivery system is incorporated in a knee joint prosthesis to prevent and to treat osteolysis and loosening of the artificial knee implant (see e.g., Agarwal, “Osteolysis—Basic Science, Incidence and Diagnosis,” Current Orthopaedics 18:220-231 (2004) and Collier et al., “Osteolysis After Total Knee Arthroplasty: Influence of Tibial Baseplate Surface Finish and Sterilization of Polyethylene Insert” J. Bone Joint Surg. 87-A: 2702-2708, (2005), which are each incorporated herein by reference). The knee joint prosthesis includes a femoral component and a tibial component which includes a tibial spacer, and a tibial tray. For example, FIG. 13 depicts an artificial joint 100 that is an artificial knee. The artificial joint 100 includes a femoral component 310 and a tibial component which includes a tibial spacer 315, and a tibial tray 320. In some embodiments, the knee joint prosthesis includes a patellar component.

The tibial spacer, the bearing surface of the tibial component, is fabricated from polyethylene. (See e.g., U.S. Patent Application No. 2005/0055101, “Endoprosthesis of the Knee and/or Other Joints” by Sifneos published Mar. 10, 2005 which is incorporated herein by reference). For example, a total knee replacement prosthesis is available from Biomet Inc., Warsaw, Ind. that includes: a femoral component cast from cobalt chrome alloy; a tibial tray cast from titanium alloy, and a tibial spacer (i.e., bearing) compression molded from high molecular weight polyethylene (see e.g., www.biomet.com/orthopedics and Xie, “A Systematic Review on Performance of the Vanguard® Complete Knee System”, published by Biomet Orthopedics, dated Jun. 30, 2011 which is incorporated herein by reference). A responsive drug delivery system is incorporated in the knee prosthesis, and includes sensors to monitor the artificial joint, a controller to process data from the sensors and a drug delivery unit which responds to signals from the controller and delivers osteogenic and anti-osteolytic drugs.

The responsive drug delivery system monitors the artificial knee joint with sensor units that are placed on internal surfaces of the joint to detect motion and load. Load sensors and motion sensors are applied or integrated into the artificial joint components. In some embodiments, the load sensors and motion sensors are present in distinct sensor units, and in some embodiments they are integrated into combined sensor units. For example, load sensors based on piezoelectric cantilevers can be placed in the tibial tray between the bone and the tibial spacer. FIG. 13 depicts sensor unit 1300 including a load sensor, positioned within the tibial tray 320.

Microcantilever sensors for detecting loads applied to orthopedic joints are described (see e.g., U.S. Pat. No. 7,097,662 “In-Vivo Orthopedic Implant Diagnostic Device for Sensing Load, Wear and Infection” issued to Evans, III et al. on Aug. 29, 2006 which is incorporated herein by reference). Microscale cantilever sensors with transmitters communicate the load forces applied to the prosthetic joint. To monitor motion, ultrasound sensors are placed in the artificial knee joint, e.g., ultrasound sensors attached to the tibial tray and to the femoral component of the prosthesis can detect and localize the surfaces of the knee joint prosthesis and associated bone. For example, pulse echo A-mode ultrasound transducers (e.g., immersion unfocused 3.5 MHz ultrasound transducers (available from Olympus Corp., Waltham, Mass.) are attached to the prosthesis to localize points on the artificial joint surfaces. FIG. 13 depicts sensor unit 1300 positioned within the tibial tray 320, including a load sensor and an ultrasound transducer. FIG. 13 also depicts sensor unit 1310 positioned within the femoral component 310. The sensor unit 310 positioned within the femoral component 310 includes a transmitter configured to send signals 1320 to the sensor unit 1300 positioned within the tibial tray 320. Sensor unit 1300 includes a receiver and circuitry for transmitting signals through the wire connector 1330 to the responsive release control unit 155. Sensor unit 1300 can, for example, transmit data regarding its own sensors, or data received in the signal 1320, through the wire connector 1330 to the responsive release control unit 155.

In addition, accelerometers may be used to track the position of each ultrasound sensor and the motion of the artificial knee joint. Moreover a fiducial marker may be implanted in the tibial plate to serve as a reference point for motion of the knee joint. Sensors to track the position and motion of bone joints are described (see e.g., U.S. Patent Application No. 2010/0198067 “Noninvasive Diagnostic System” by Mahfouz et al. published Aug. 5, 2010 and U.S. Pat. No. 5,533,519 “Method and Apparatus for Diagnosing Joints” issued to Radke et al. on Jul. 9, 1996 which are incorporated herein by reference). Signals from the motion sensors and the load sensors are transmitted wirelessly to a control unit. Data on the number of motion cycles and load forces on the knee joint prosthesis are received by a control unit (e.g., circuitry, microprocessors) and processed to determine the accumulated load and the number of motion cycles the prosthesis has experienced. For example the accumulated motion cycles and axial load values may be: 1000 motion cycles at an average load of 1600 N (Newton=Force to accelerate 1 kilogram at 1 meter/second²), yielding a load×motion product of 1.6×10⁶ N-cycles. The controller circuitry compares the load×motion cycles to predetermined limits for the knee joint prosthesis. If preset limits in load×motion are equaled or exceeded the control unit signals to the drug delivery unit to deliver drugs to the prosthesis to reduce and prevent osteolysis.

The responsive drug delivery system delivers drugs to internal sites in the artificial knee implant to reduce osteolysis and prevent loosening of the knee prosthesis. The drug delivery system actively delivers drug in response to the controller. For example, the knee prosthesis may include multiple microreservoirs (approximately 100 μL in volume) which are incorporated in the tibial tray with their openings facing the tibia bone and thus facilitating drug delivery to the prosthesis/bone interface. See, for example, reservoir 165 in FIG. 13, positioned to release medicament shown by dotted arrows into the region adjacent to the tibial component at a position facing the patella. The reservoirs may be capped with electrothermally activated caps. For example, thin metal films composed of conductive gold or gold-tin cap the reservoirs. The caps are connected to circuitry and a power source or battery which provides electric current. Electronic activation of the reservoir caps by the controller results in thermal ablation of the caps and drug release by individual microreservoirs. For example, microreservoirs with electronic control of drug release are described for orthopedic devices (see e.g., U.S. Patent Application No. 2007/0016163 “Medical and Dental Implant Devices for Controlled Drug Delivery,” by Santini, Jr. et al. published on Jan. 18, 2007 which is incorporated herein by reference). Multiple drug delivery units with approximately 100 microreservoirs each may be incorporated in the tibial tray and the femoral component of the knee prosthesis with circuitry providing electric current to each reservoir cap. Thermal ablation of selected reservoir caps is initiated by the controller in response to sensor data on motion cycles and load experienced by the artificial joint. The controller activates release of osteogenic factors to promote bone growth at the implant/bone interface. For example, selected reservoirs may contain bone morphogenetic protein (BMP), e.g., BMP-2, and transforming growth factor-β(TGF-β) which promote growth of bone tissue (see e.g., U.S. Patent Application No. 2007/0016163, Ibid.). Simultaneously or sequentially the controller may respond to joint motion and load by activating reservoirs which release anti-osteolytic agents. For example, selected reservoirs may contain an adenosine receptor agonist which inhibits osteoclast differentiation and reduces bone resorption. An adenosine receptor agonist, CGS21680, effective at concentrations of approximately 0.1 nM to 1.0 μM in reducing bone resorption and artificial joint loosening is described (see e.g., U.S. Pat. No. 8,183,225 “Inhibition of Bone Resorption Using Medical Implants Containing Adenosine Receptor Antagonists” issued to Cronstein et al. on May 22, 2012 which is incorporated herein by reference). Signals from the controller may ablate one or more reservoir caps and release the adenosine agonist, CGS21680 into the prosthesis/bone interface to reduce and prevent bone resorption and joint loosening.

Continuous data collection on the motion and load experienced by the knee joint prosthesis is processed by the controller, and cumulative data are compared to preset standards for bone resorption and joint loosening. Periodically the controller signals the drug delivery unit to deliver additional doses of osteogenic and/or anti-osteolytic agents.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system for responsive release of a medicament in an artificial joint region, comprising:
an implantable sensor unit including at least one sensor, the implantable sensor unit configured to be implanted in an artificial joint region;
a responsive release control unit including an electronic controller and memory, the responsive release control unit configured to receive signals from the implantable sensor unit and to send signals to an implantable medicament release unit; and
the implantable medicament release unit, including a reservoir and a controllable release unit attached to the reservoir, the controllable release unit configured to provide access to the reservoir in response to signals from the responsive release control unit, the implantable medicament release unit configured to be implanted in the artificial joint region.

2-3. (canceled)

4. The system of claim 1, wherein the implantable sensor unit including at least one sensor comprises:
a motion sensor.

5. The system of claim 1, wherein the implantable sensor unit including at least one sensor comprises:
a load sensor.

6. The system of claim 1, wherein the implantable sensor unit including at least one sensor comprises:
a motion sensor; and
a load sensor.

7. The system of claim 1, wherein the implantable sensor unit including at least one sensor comprises:
a position sensor, configured to detect a position of the artificial joint.

8. The system of claim 1, wherein the implantable sensor unit including at least one sensor comprises:
a distance sensor, configured to detect a distance of the artificial joint.

9. The system of claim 1, wherein the implantable sensor unit including at least one sensor further comprises:
a transmitter.

10. (canceled)

11. The system of claim 1, wherein the responsive release control unit further comprises:
a receiver; and
a signal processor.

12. The system of claim 1, wherein the responsive release control unit further comprises:
a transmitter; and
a signal processor.

13. The system of claim 1, wherein the responsive release control unit further comprises:
an antenna.

14. (canceled)

15. The system of claim 1, wherein the controllable release unit of the implantable medicament release unit comprises:
one or more electrical pulse generators.

16. The system of claim 1, wherein the controllable release unit of the implantable medicament release unit comprises:
one or more optical pulse generators.

17. (canceled)

18. The system of claim 1, wherein the implantable sensor unit is configured to attach to a first location on a first component of the artificial joint, and wherein the implantable medicament release unit is configured to attach to a second location on a second component of the artificial joint.

19. The system of claim 1, further comprising:
a fiducial marker configured to be attached to the joint at a first location on a first component of the artificial joint;
and wherein the implantable sensor unit is configured to be attached to the artificial joint at a second location on a second component and the at least one sensor includes a sensor configured to detect a relative position of the fiducial marker.

20. A system for responsive release of a medicament in an artificial joint, comprising:
at least one fiducial unit configured to attach to a first region of a first component of an artificial joint;
an implantable sensor unit configured to attach to a second region of a second component of the artificial joint, the implantable sensor unit including at least one sensor configured to sense a position relative to the at least one fiducial unit;
a responsive release control unit including an electronic controller and memory, the responsive release control unit configured to receive signals from the implantable sensor unit and to send signals to an implantable medicament release unit; and
the implantable medicament release unit, including at least one reservoir, and at least one controllable release unit configured to respond to signals from the responsive release control unit.

21-22. (canceled)

23. The system of claim 20, wherein the at least one fiducial unit is configured to be affixed to the first region of the first component of the artificial joint.

24. The system of claim 20, wherein the at least one fiducial unit is integral to the first region of the first component of the artificial joint.

25. The system of claim 20, wherein the implantable sensor unit is configured to be affixed to the second region of the second component of the artificial joint.

26. The system of claim 20, wherein the implantable sensor unit is integral to the second region of the second component of the artificial joint.

27. The system of claim 20, wherein the implantable sensor unit including at least one sensor comprises:
a motion sensor.

28. The system of claim 20, wherein the implantable sensor unit further comprises:
a distance sensor, configured to detect a distance relative to the at least one fiducial unit.

29. The system of claim 20, wherein the implantable sensor unit further comprises:
a load sensor.

30. The system of claim 20, wherein the implantable sensor unit further comprises:
a transmitter.

31. The system of claim 20, wherein the implantable sensor unit further comprises:
a receiver.

32. (canceled)

33. The system of claim 20, wherein the responsive release control unit further comprises:
a transmitter.

34. The system of claim 20, wherein the responsive release control unit further comprises:
a receiver; and
a signal processor.

35. The system of claim 20, wherein the responsive release control unit further comprises:
an antenna.

36. (canceled)

37. The system of claim 20, wherein the controllable release unit of the implantable medicament release unit comprises:
one or more electrical pulse generators.

38. The system of claim 20, wherein the controllable release unit of the implantable medicament release unit comprises:
one or more optical pulse generators.

39. (canceled)

40. The system of claim 20, comprising:
a plurality of fiducial units, each fiducial unit configured to attach to the first region of the first component of the artificial joint; and
wherein the implantable sensor unit includes at least one sensor configured to sense the position of each of the plurality of fiducial units.

41. A responsive release control unit for a medicament in an artificial joint, comprising:
circuitry configured to accept information relating to motion of an artificial joint in an individual;
circuitry configured to save the accepted information in memory within an artificial joint motion history;
circuitry configured to form a comparison of the artificial joint motion history with preset parameters of the motion of the artificial joint for the individual;
circuitry configured to determine if the comparison exceeds a preset limit for the individual; and
circuitry configured to send a signal to an implanted medicament release unit in response to the determination.

42. (canceled)

43. The responsive release control unit of claim 41, wherein the circuitry configured to accept information relating to motion of an artificial joint comprises:
circuitry configured to accept information regarding a load on the artificial joint during motion.

44. The responsive release control unit of claim 41, wherein the circuitry configured to accept information relating to motion of an artificial joint comprises:
circuitry configured to accept information regarding repetition of motion of the artificial joint.

45. The responsive release control unit of claim 41, wherein the circuitry configured to accept information relating to motion of an artificial joint comprises:
circuitry configured to accept information regarding extension of the artificial joint.

46. (canceled)

47. The responsive release control unit of claim 41, wherein the circuitry configured to form a comparison of the artificial joint motion history with preset parameters of the motion of the artificial joint for the individual comprises:
circuitry configured to compare information regarding a number of movements of the artificial joint.

48. The responsive release control unit of claim 41, wherein the circuitry configured to form a comparison of the artificial joint motion history with preset parameters of the motion of the artificial joint for the individual comprises:
circuitry configured to compare information regarding extension of the artificial joint.

49. The responsive release control unit of claim 41, wherein the circuitry configured to form a comparison of the artificial joint motion history with preset parameters of the motion of the artificial joint for the individual comprises:
circuitry configured to compare information regarding a cumulative load on the artificial joint.

50. The responsive release control unit of claim 41, wherein the circuitry configured to determine if the comparison exceeds a preset limit for the individual comprises:
circuitry operating a look-up table.

51. (canceled)

52. The responsive release control unit of claim 41, wherein the circuitry configured to send a signal to an implanted medicament release unit in response to the determination comprises:
circuitry configured to send a wireless signal.

53. The responsive release control unit of claim 41, wherein the circuitry configured to send a signal to an implanted medicament release unit in response to the determination comprises:
circuitry configured to send a signal through a wire connection.

54. The responsive release control unit of claim 41, wherein the circuitry configured to send a signal to an implanted medicament release unit in response to the determination comprises:
circuitry configured to send a release signal.

55. The responsive release control unit of claim 41, further comprising:
circuitry configured to send a signal to a user interface in response to the determination.

56-57. (canceled)
